(19) United States
(12) Patent Application Publication
(10) Pub. No.: US 2026/0146993 A1
(43) Pub. Date: May 28, 2026

(54) METHODS AND APPARATUSES FOR DETECTING BIOMOLECULE AND ANALYTE INTERACTIONS

(71) Applicant: Seer, Inc., Redwood City, CA (US)

(72) Inventors: Daniel HORNBURG, Foster City, CA (US); Shadi ROSHDIFERDOSI, San Carlos, CA (US); Brittany LEE, San Carlos, CA (US); Moaraj HASAN, San Francisco, CA (US)

(21) Appl. No.: 19/255,568

(22) Filed: Jun. 30, 2025

Related U.S. Application Data
(63) Continuation of application No. PCT/US2023/085783, filed on Dec. 22, 2023.
(60) Provisional application No. 63/477,707, filed on Dec. 29, 2022.

(51) Int. Cl. G01N 33/543; C12Q 1/34; G01N 33/68

(52) U.S. Cl. CPC G01N 33/54326; C12Q 1/34; G01N 33/6845; G01N 33/6848; G01N 2333/95

(57) ABSTRACT

Disclosed herein are methods for identifying biomolecule-analyte interactions in vitro and in subjects, and apparatuses for the same. In one aspect, provided herein are computer systems for analyzing the same.

### CROSS-REFERENCE

This application is a continuation of International Patent Application No. PCT/US2023/085783, filed Dec. 22, 2023, which claims the benefit of U.S. Provisional Application No. 63/477,707, filed Dec. 29, 2022, which application is incorporated herein by reference in its entirety.

### BACKGROUND

Analysis of biomolecule interactions with ligands typically require implementing laborious methods, rely finely on the parameters of the method, require inducing precipitation, which may not be feasible for all proteins or ligands, and is irreversible. While some recent progress has improved analysis in regards to some of these limitations, improvement for all of these limitations is needed for detecting a broader range of biomolecule and analyte interactions. The present disclosure provides systems and methods thereof to address this need.

### SUMMARY

In one aspect, described herein is a method for identifying a biomolecule-analyte interaction, the method comprising: (a) contacting a surface with a first composition comprising first concentrations of a plurality of different biomolecules and a first concentration of one or more analytes to form one or more first biomolecule coronas; (b) assaying the one or more first biomolecule coronas and/or a supernatant of the first composition to obtain a first assay data representing quantities of the plurality of different biomolecules in the one or more first biomolecule coronas and/or the supernatant of the first composition; (c) providing a second assay data representing quantities of the plurality of different biomolecules in one or more second biomolecule coronas and/or a supernatant of a second composition; and (d) identifying a biomolecule-analyte interaction based on one or more variations between the first assay data and the second assay data.

In some embodiments, the method further comprises, (e) identifying structural changes in the biomolecule which are a result of the biomolecule-analyte interaction.

In some embodiments, in (c), the second composition comprises the first concentrations of the plurality of different biomolecules and optionally a second concentration of one or more analytes.

In some embodiments, the second composition comprises the first concentrations of the plurality of different biomolecules and no concentration of the one or more analytes.

In some embodiments, wherein the second composition comprises the first concentrations of the plurality of different biomolecules and a second concentration of one or more analytes.

In some embodiments, in (c), the one or more second biomolecule coronas and the supernatant of the second composition are obtained by contacting the second composition with a second surface.

In some embodiments, in (c), the first surface and the second surface have the same physicochemical properties.

In some embodiments, the physicochemical properties are selected from the group consisting of composition, core material, shell material, porosity size, surface charge, hydrophobicity, hydrophilicity, surface functionality, surface topography, surface curvature, shape, or any combination thereof.

In some embodiments, in (c), the first surface and the second surface have the same physicochemical properties, wherein the physicochemical properties are selected from the group consisting of composition, core material, shell material, porosity size, surface charge, hydrophobicity, hydrophilicity, surface functionality, surface topography, surface curvature, shape, or any combination thereof.

In some embodiments, in (c), the first concentration and the second concentration are different.

In some embodiments, the second assay data is retrieved from a database. In some embodiments, the second assay data is acquired experimentally.

In some embodiments, (c) is performed prior to (b).

In some embodiments, (c) is performed prior to (a)

In some embodiments, (c) and (a) are performed, at least in part, during the same time.

In some embodiments, the plurality of different biomolecules comprise a nucleic acid, a small molecule, a protein, a lipid, a polysaccharide, a metabolite, or any combination thereof.

In some embodiments, a biomolecule of the plurality of different biomolecules is a nucleic acid, a small molecule, a protein, a lipid, a polysaccharide, or a metabolite. In some embodiments, the plurality of different biomolecules comprise proteins. In some embodiments, the plurality of different biomolecules are proteins. In some embodiments, the biomolecule is a protein. In some embodiments, the protein is a shuttle protein, a ligand, a member of a protein complex, comprises a low complexity region, comprises an interaction domain, involved in signal transduction, or any combination thereof.

In some embodiments, the one or more first biomolecule coronas comprise one or more different biomolecules. In some embodiments, the one or more first biomolecule coronas comprise the one or more analytes.

In some embodiments, the plurality of different biomolecules adsorb onto the surface to form the one or more first biomolecule coronas.

In some embodiments, the one or more analytes adsorb onto the surface to form the one or more first biomolecule coronas.

In some embodiments, the surface is a particle or bead. In some embodiments, the surface is a particle. In some embodiments, the particle is a micelle, liposome, iron oxide particle, silver particle, gold particle, palladium particle, quantum dots, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, and 2-(3-aminopropylamino)ethanol particle, protein functionalized particle, ubiquitin functionalized particle, polysaccharide coated particle, or dextran functionalized particle.

In some embodiments, the particle is a nanoparticle or a microparticle. In some embodiments, the particle is a magnetic particle. In some embodiments, the magnetic particle is a superparamagnetic iron oxide particle. In some embodiments, the particle comprises iron oxide material. In some embodiments, the particle comprises an iron oxide core. In some embodiments, the particle has iron oxide crystals embedded in a polystyrene core. In some embodiments, the particle comprises a polymer coating. In some embodiments, the particle comprises a carboxylated polymer, an aminated polymer, a zwitterionic polymer, or any combination thereof. In some embodiments, the particle comprises a polymer coating, wherein the polymer coating comprises a carboxylated polymer, an aminated polymer, a zwitterionic polymer, or any combination thereof. In some embodiments, the particle comprises an iron oxide core with a silica shell coating. In some embodiments, the particle comprises an iron oxide core with a poly(N-(3-(dimethylamino)propyl) methacrylamide) (PDMAPMA) coating. In some embodiments, the particle comprises an iron oxide core with a poly(oligo(ethylene glycol) methyl ether methacrylate) (POEGMA) coating. In some embodiments, the particle comprises an iron oxide core with a poly(N-(3-(dimethylamino)propyl) methacrylamide) (PDMAPMA) coating or a poly(oligo(ethylene glycol) methyl ether methacrylate) (POEGMA) coating. In some embodiments, the particle comprises a positive surface charge. In some embodiments, the particle comprises a negative surface charge. In some embodiments, the particle comprises a neutral surface charge.

In some embodiments, a biomolecule corona of the one or more first biomolecule coronas or the one or more second biomolecule coronas comprises: a first physicochemical property selected from the group consisting of a magnetic core, a polystyrene core, a metal core, a gold core, a metal oxide core, an iron oxide core, a polymeric core, and a silica core; and a second physicochemical property selected from the group consisting of a carboxylated surface, an amino surface, a silica surface, a polymer surface, a phosphate sugar functionalized surface, a phenol functionalized surface, a citrate functionalized surface, a Jeffamine surface, and a silica silanol surface.

In some embodiments, the analyte is provided in a biological sample. In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. Wherein the biological sample comprises proteins or protein groups. In some embodiments, the analyte is provided in a biological sample, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises at least 100 proteins or protein groups. In some embodiments, the biological sample comprises at least 500 proteins or protein groups. In some embodiments, the biological sample comprises at least 1000 proteins or protein groups.

In some embodiments, the analyte is not provided in a biological sample.

In some embodiments, the analyte is a drug, therapeutic agent, nutrient, biomolecule, pathogen, or a metabolite thereof. In some embodiments, the drug comprises an antibody, a biotherapeutic, or a chemotherapeutic biomolecule.

In some embodiments, the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof.

In some embodiments, the pathogen comprises a Hepatitis B virus, a Hepatitis C virus, a COVID-19 virus, or a HIV.

In some embodiments, the analyte is a non-naturally occurring small molecule.

In some embodiments, the small molecule comprises a drug.

In some embodiments, the analyte is a biomolecule.

In some embodiments, the biomolecule is a protein or a portion thereof.

In some embodiments, the protein comprises a receptor or enzyme configured to bind to the biomolecule coupled to the one or more distinct biomolecule coronas.

In some embodiments, the protein comprises a peptide sequence having a minimum length of 7 amino acid residues.

In some embodiments, the biomolecule is a lipid.

In some embodiments, the biomolecule is metabolite.

In some embodiments, the analyte is configured to interact with one or more different biomolecules.

In some embodiments, the biomolecule of the one or more different biomolecules is a protein.

In some embodiments, the analyte interacts with an allosteric site on the protein. In some embodiments, the analyte interacts with an active site on the protein. In some embodiments, the analyte interacts with a target receptor site on the protein.

In some embodiments, the interaction between a biomolecule of the one or more different biomolecules and the analyte comprises a coulombic interaction, a hydrogen bond, a Van der Waals interaction, solvent exclusion, hydrophobic effect, or a combination thereof.

In some embodiments, the analyte comprises two or more binding moieties.

In some embodiments, a first binding moiety of the two or more binding moieties forms a complex with a first biomolecule of the plurality of different biomolecules and a second binding moiety of the two or more binding moieties forms a complex with a second biomolecule of the plurality of different biomolecules.

In some embodiments, the first biomolecule and the second biomolecule are different.

In some embodiments, a first binding moiety of the two or more binding moieties forms a first complex with a first component of the biomolecule of the one or more distinct biomolecule coronas and a second binding moiety of the two or more binding moieties forms a second complex with a second component of the biomolecule.

In some embodiments, the first component and the second component are different.

In some embodiments, the first complex and the second complex are in equilibrium.

In some embodiments, the analyte forms a complex with only one biomolecule of the plurality of different biomolecules.

In some embodiments, each of the two or more binding moieties simultaneously form a complex with the first biomolecule and the second biomolecule of the plurality of different biomolecules.

In some embodiments, the biomolecule-analyte interaction is direct.

In some embodiments, the biomolecule-analyte interaction is indirect.

In some embodiments, the biomolecule-analyte interaction comprises an electrostatic interaction between the biomolecule and the analyte. In some embodiments, the electrostatic interaction comprises an ionic bond between the biomolecule and the analyte. In some embodiments, the electrostatic interaction comprises a hydrogen-bond between the biomolecule and the analyte. In some embodiments, the electrostatic interaction comprises Van der Waals forces.

In some embodiments, the electrostatic interaction is reversible.

In some embodiments, in (a) contacting comprises incubating the one or more first biomolecule coronas and the analyte.

In some embodiments, incubating is performed at a temperature of about 0 degrees Celsius to about 90 degrees Celsius. In some embodiments, incubating is performed for a length of time of about 30 seconds to about 12 hours.

In some embodiments, in (a) contacting comprises adjusting a pH of a mixture comprising the one or more first biomolecule corona.

In some embodiments, the pH is adjusted such that a component of a biomolecule of the plurality of different biomolecules is inhibited from forming a biomolecule-analyte interaction.

In some embodiments, prior to (b) and subsequent to (a), the method comprises isolating the one or more first biomolecule coronas and/or the supernatant.

In some embodiments, isolating comprises magnetically isolating the one or more first biomolecule coronas.

In some embodiments, prior to (b) and subsequent to (a), the method comprises isolating the one or more first biomolecule coronas and/or the supernatant, wherein isolating comprises magnetically isolating the one or more first biomolecule coronas.

In some embodiments, in (b) assaying comprises a denaturing and/or chemical treatment.

In some embodiments, denaturing and/or chemical treatment comprises heating, reduction, alkylation, protease digestion, or combination thereof.

In some embodiments, in (b) assaying comprises identifying protein groups. The In some embodiments, the assaying is capable of identifying 1 to 20,000 protein groups. In some embodiments, at least 100 protein groups are identified.

In some embodiments, assaying comprises centrifuging the supernatant comprising the biomolecule-analyte interaction.

In some embodiments, assaying comprises detecting at least one of the one or more first biomolecule coronas or the biomolecule-analyte interactions in a sample through Mass Spectrometry.

In some embodiments, the assaying comprises detecting at least one of the one or more first biomolecule coronas or the biomolecule-analyte interactions in a sample using high throughput single molecule protein sequencing.

In some embodiments, in (b) assaying comprises quantifying at least one of the one or more first biomolecule coronas or the biomolecule-analyte interactions in a sample through Mass Spectrometry.

In some embodiments, in (b) assaying comprises digesting proteins in the protein corona using a non-selective proteinase.

In some embodiments, the non-selective protease is Proteinase K.

In some embodiments, in (b) assaying comprises cross-linking proteins in the protein corona and detecting cross-link proteins.

In some embodiments, the detecting comprises cross-linking mass spectrometry. In some embodiments, the cross-linking is performed with a mass spectrometry cleavable cross-linking agent.

In some embodiments, the method further comprises repeating incubating, isolating, and assaying.

In some embodiments, the incubating, the isolating, and the assaying yields a percent quantile normalized coefficient (QNCV) of variation of 30% or less, as determined by comparing a mass spectrometry feature.

In some embodiments, the incubating, the isolating, and the assaying yields a percent quantile normalized coefficient (QNCV) of variation of 20% or less, as determined by comparing a mass spectrometry feature.

In some embodiments, the method further comprises (e) detecting variations across one or more samples.

In some embodiments, detecting variations comprises detecting a change in a composition of the supernatant in comparison to an original composition of the supernatant.

In some embodiments, the original composition of the supernatant comprises the surface with the first composition comprising first concentrations of the plurality of different biomolecules without contact with the analyte.

In some embodiments, subsequent to (a) the supernatant comprises the biomolecule-analyte interaction.

In some embodiments, detecting variations comprises detecting a change in a composition of the one or more second biomolecule coronas in comparison to a composition of the one or more first biomolecule coronas.

In some embodiments, the composition of the one or more second biomolecule coronas comprises the plurality of different biomolecules without contact with the analyte.

In some embodiments, subsequent to (a) the one or more first biomolecule coronas is altered such that a biomolecule of the biomolecule-analyte interaction is absent from at least one of one or more first biomolecule coronas.

In some embodiments, detecting variations across one or more samples comprises detecting a change in a composition of the supernatant in comparison to an original composition of the supernatant and detecting a change in a composition of the one or more distinct biomolecule coronas in comparison to an original composition of the one or more distinct biomolecule coronas.

In some embodiments, the second composition of the supernatant comprises the one or more biomolecule coronas without contact with the analyte.

In some embodiments, subsequent to (a) the supernatant comprises the biomolecule-analyte interaction.

In some embodiments, the original composition of the one or more distinct biomolecule coronas comprises the one or more biomolecule coronas without contact with the analyte.

In some embodiments, subsequent to (a) the one or more distinct biomolecule coronas is altered such that a biomolecule of the biomolecule-analyte interaction is absent from at least one of one or more distinct biomolecule coronas.

In some embodiments, identifying the biomolecule-analyte interaction comprises identifying a biological state.

In some embodiments, the biological state is a healthy biological state.

In some embodiments, the biological state is a disease biological state.

In some embodiments, identifying the biomolecule-analyte interaction comprises identifying a biological state, wherein the biological state is a healthy biological state or a disease biological state.

In some embodiments, identifying the biomolecule-analyte interaction comprises identifying a signal transduction pathway.

In some embodiments, a thermodynamic stability of the biomolecule-analyte interaction is greater than a thermodynamic stability of an interaction comprising the analyte and the surface with a first composition such that the biomolecule-analyte interaction decouples from the one or more first biomolecule coronas, and wherein a composition of the one or more first biomolecule coronas is altered.

In some embodiments, the biomolecule-analyte interaction decouples from the one or more first biomolecule coronas spontaneously. In some embodiments, the biomolecule-analyte interaction decouples from the one or more first biomolecule coronas upon induction of a stimuli.

In some embodiments, the stimuli is a change in chemical environment, light, temperature, pressure, electricity, or a combination thereof.

In some embodiments, the change in chemical environment comprises a change in pH, a change in concentration, an additional of a denaturing agent, or a combination thereof.

In some embodiments, the denaturing agent comprises GuHCl, urea, SDS, or a combination thereof.

In some embodiments, upon decoupling, the biomolecule-analyte interaction is a part of the supernatant.

In some embodiments, the method comprises contacting two or more biomolecule coronas with the analyte, wherein each distinct biomolecule corona of the two or more distinct biomolecule coronas differs from each other by one or more physicochemical properties.

In some embodiments, the one or more physicochemical properties are selected from the group consisting of: composition, size, surface charge, hydrophobicity, hydrophilicity, surface functionality, surface topography, surface curvature, shape, and any combination thereof.

In some embodiments, in (b), the method comprises contacting two or more analytes with the biomolecule coronas, wherein each analyte of the two or more analyte is different.

In some embodiments, each analyte of the two or more analyte forms a complex with a biomolecule coupled to the surface.

In another aspect, described herein is an apparatus for identifying biomolecule-analyte interactions, the apparatus comprising: a substrate comprising a plurality of partitions, wherein the plurality of partitions comprises plurality of different biomolecules contacted with a surface to form one or more first biomolecule corona; a sample storage unit comprising the first biomolecule corona; and a loading unit that is movable at least across the substrate; wherein the apparatus is programmed to perform a series of steps comprising: contacting a biological sample with a specified partition of the sensor array; incubating the biological sample with an analyte contained within the partition of the sensor array plate to form a biomolecule corona; removing a supernatant from a partition; and optionally preparing biomolecules from the biomolecule corona for analysis.

In some embodiments, the apparatus is automated.

In some embodiments, the one or more volumes of an analyte comprise one or more concentrations of the analyte.

In some embodiments, the substrate is a multi-well plate.

In some embodiments, the loading unit comprises a plurality of pipettes.

In some embodiments, the loading unit is configured to dispense a volume of 5 μL to 1000 μL of a solution into one or more partitions of the plurality of partitions.

In some embodiments, the volume of a solution dispensed into each of the one or more partitions of the plurality of partitions is different.

In some embodiments, the analyte comprises protein.

In some embodiments, the analyte comprises a drug.

In some embodiments, the second biomolecule corona is assayed.

In some embodiments, the supernatant is assayed.

In another aspect, described herein is a computer-based system for identifying a biomolecule-analyte interaction, the system comprising: a processor, and a non-transitory computer readable medium comprising a software configured to cause the processor to: (i) receive an assay data from the non-transitory computer readable medium, wherein the assay data comprises biomolecule information for a plurality of different biomolecule coronas and supernatants from a sample, wherein the plurality of different biomolecule coronas comprise a plurality of biomolecules and a surface, and wherein the different biomolecule coronas are contacted with a different concentration of an analyte; and (ii) performing a statistical analysis on the assay data to identify the at least one biomolecule-analyte interaction.

In some embodiments, the concentration of an analyte is at least 1 ppb.

In some embodiments, the identifying is completed at multiple concentrations to measure a binding constant for the biomolecule-analyte interaction.

In some embodiments, the biomolecule is a protein. In some embodiments, the analyte is a protein. In some embodiments, the analyte is a drug.

In some embodiments, the drug is an antibody, a biotherapeutic, or a chemotherapeutic biomolecule.

In some embodiments, the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof.

In some embodiments, the surface is a particle or bead. In some embodiments, the surface is a particle.

In another aspect, described herein is a method of identifying biomolecule-analyte interactions in a subject, the method comprising: (a) contacting a first surface with a first composition comprising concentrations of a plurality of different biomolecules and a first concentration of one or more analytes to form one or more first biomolecule corona, wherein the plurality of different biomolecules are derived from a biological sample of the subject; (b) assaying the one or more first biomolecule coronas and/or a supernatant of the first composition to obtain a first assay data representing quantities of the biomolecules in the first biomolecule coronas and/or the supernatant of the first composition; (c) contacting a second surface with a second composition comprising concentrations of a plurality of different biomolecules and a second concentration of one or more analytes to form one or more second biomolecule corona, wherein the plurality of different biomolecules are derived from the biological sample of the subject; (d) assaying the one or more second biomolecule coronas and/or a supernatant of the second composition to obtain a second assay data representing quantities of the biomolecules in the second biomolecule coronas and/or the supernatant of the second composition; and (e) identifying biomolecules that interact with the analytes based on variations between the first assay data and second assay data.

In some embodiments, the first composition and the second composition are different.

In some embodiments, the first concentration of the one or more analytes is different from the second concentration of the one or more analytes.

In some embodiments, the first surface and the second surface are the same.

In some embodiments, the first surface and the second surface is a particle or bead.

In some embodiments, the surface is a particle. In some embodiments, the particle is a nanoparticle or a microparticle. In some embodiments, the particle is a magnetic particle. In some embodiments, the magnetic particle is a superparamagnetic iron oxide particle. In some embodiments, the particle comprises iron oxide material. In some embodiments, the particle has iron oxide crystals embedded in a polystyrene core. In some embodiments, the particle comprises a polymer coating. In some embodiments, the particle comprises a carboxylated polymer, an aminated polymer, a zwitterionic polymer, or any combination thereof. In some embodiments, the particle comprises an iron oxide core with a silica shell coating. In some embodiments, the particle comprises an iron oxide core with a poly(N-(3-dimethylamino)propyl) methacrylamide) (PDMAPMA) coating. In some embodiments, the particle comprises an iron oxide core with a poly(oligo(ethylene glycol) methyl ether methacrylate) POEGMA) coating.

In some embodiments, the particle comprises a positive surface charge. In some embodiments, the particle comprises a negative surface charge. In some embodiments, the particle comprises a neutral surface charge.

In some embodiments, the analyte is a drug or a therapeutic agent.

In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample is a biofluid. In some embodiments, the biological sample is plasma or serum.

Also provided herein is a method comprising: (a) incubating a biofluid with a surface and an analyte to form a protein corona on the surface; (b) analyzing the proteins from the protein corona using mass spectrometry to obtain a protein corona signature associated with the biofluid and analyte; and (c) comparing the protein corona to a suitable control protein corona signature to identify at least one protein-analyte interaction.

In some embodiments, the biofluid is plasma or serum.

In some embodiments, the surface comprises a plurality of particles.

In some embodiments, incubating the biofluid with the surface and the analyte comprises incubating for at least 20 minutes. In some embodiments, incubating the biofluid with the surface and the analyte comprises incubating at a temperature of 15° C. to 75° C.

In some embodiments, the proteins from the protein corona are digested before analysis.

In some embodiments, the method further comprises separating the protein corona dn surface from a supernatant before analyzing the protein corona.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an example of a schematic illustrating how a particle with various biomolecules interacting with the surface of the particle may be affected upon exposure to one or more analytes.

FIG. 2 shows an example of how ligand (or analyte) dose affects a quantity of biomolecules released from a biomolecule corona.

FIG. 3 shows an example of a computer system that is programmed or otherwise configured to implement a method for identifying a biomolecule-analyte interaction.

### DETAILED DESCRIPTION

While various embodiments of the disclosure have been shown and described herein, it will be obvious to those skilled in the art that such embodiments are provided by way of example only. Numerous variations, changes, and substitutions can occur to those skilled in the art without departing from the disclosure. It should be understood that various alternatives to the embodiments of the disclosure described herein can be employed. The section headings used herein are for organizational purposes only and are not to be construed as limiting the subject matter described.

Previously, thermal shift assays have been used that leverage change in protein stability upon binding of ligands, like small molecules and drugs, to heat induced precipitation. In these thermal shift assays, protein precipitated at different temperatures can be analyzed via Mass Spectrometry. However, thermal shift assays require labororious separation of precipitated proteins by centrifugation and the resolution largely depends on the time, temperature, and gradient or stepping applied. In addition, thermal shift assays require induced insolubility in order to assay binding events which is inefficient for highly stable proteins, very small proteins, and small molecules, like lipids or metabolites Moreover, denaturation is often irreversible and limited in its capability to evaluate dynamically shifting equilibria. In addition, because thermal shift assays operate at non-physiological conditions, they cannot assay, for example, active secretion systems of cells and tissues in situ.

The methods and systems disclosed herein may leverage phase-partitioning on surfaces to improve upon one or more of the issues relating to current thermal shift assays. Surfaces may separate biomolecules based on their affinity to the surface. The methods and systems disclosed herein may allow for analytes to bind to a broader range of biomolecules and the interactions may be measured by analyzing a change in the corona composition and/or a supernatant. Additionally, the systems and methods of the disclosure may enable a more sensitive assessment of interactions between a biomolecule and analyte by the capacity of engineered NPs to more deeply sample the dynamic range of proteomes and may enable more efficient monitoring of interactions due to assaying different parameters (e.g., temperature, concentration, etc.) in the same sample.

### Definitions

Unless defined otherwise, all technical and scientific terms used herein have the same meaning as is commonly understood by one of skill in the art to which this disclosure belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Whenever the term “at least,” “greater than” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” as used herein referring to a number or a numerical range means that the number or numerical range referred to is an approximation within experimental variability (or within statistical experimental error), and thus the number or numerical range may vary between 1% and 15% of the stated number or numerical range.

As used in this specification and the appended claims, the singular forms “a,” “an,” “and” “the” include plural referents unless the content clearly dictates otherwise. It should also be noted that the term “or” is generally employed in its sense including “and/or” unless the content clearly dictates otherwise.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals.

As used herein, the term “biomolecule corona” refers to the plurality of different biomolecules that are able to bind to a surface. The term “biomolecule corona” encompasses “protein corona” which is a term used in the art to refer to the proteins, lipids, and other plasma components that bind nanoparticles when they come into contact with biological samples or biological systems.

As used herein, the term “biomolecule corona signature” refers to the composition, signature, or pattern of different biomolecules that are bound to each separate surface. The signature not only refers to the different biomolecules but also the differences in the amount, level or quantity of the biomolecule bound to the surface, or differences in the conformational state of the biomolecule that is bound to the surface. It is contemplated that the biomolecule corona signatures of surface may contain some of the same biomolecules, may contain distinct biomolecules with regard to other surfaces, and/or may differ in level or quantity, type, or conformation of the biomolecule. The biomolecule corona signature may depend on not only the physicochemical properties of surface, but also the nature of the sample and the duration of the exposure. In some cases, the biomolecule corona signature is a protein corona signature. In yet another case, the biomolecule corona signature is a metabolite corona signature. In some cases, the biomolecule corona signature is a lipidomic corona signature.

### Methods

Provided herein are methods for identifying a biomolecule-analyte interaction comprising: (a) contacting a surface with a first composition comprising first concentrations of a plurality of different biomolecules and a first concentration of one or more analytes to form one or more first biomolecule coronas; (b) assaying the one or more first biomolecule coronas and/or a supernatant of the first composition to obtain a first assay data representing quantities of the plurality of different biomolecules in the one or more first biomolecule coronas and/or the supernatant of the first composition; (c) providing a second assay data representing quantities of the pluraliy of different biomolecules in one or more second biomolecule coronas and/or a supernatant of a second composition; and (d) identifying a biomolecule-analyte interaction based on or more variations between the first assay data and the second assay data.

In some embodiments, identifying a biomolecule-analyte interaction based on one or more variations between first assay data and second assay data may comprise using statistical analyses. Statistical analysis may be used to identify significant deviations which may be used to identify biomolecule-analyte interactions. For example, analysis using the Student's t-test may be used to identify statistically significant variations in the assay data that can be associated with the analyte. This may be, in some embodiments, if the relative abundance measured in the corona or supernatant changes by for example 1.2×, 1.5×, 2×, 4×, 10×, 100× significantly (p-value<than 5%).

In some embodiments, the method may further comprise identifying structural changes in the biomolecule which are a result of the biomolecule-analyte interaction. In some embodiments, the second composition may comprise the first concentrations of the plurality of different biomolecules, and optionally a second concentration of the one or more analytes. In some embodiments, the second composition may comprise the first concentrations of the plurality of different biomolecules and a second concentration of the one or more analytes. In some embodiments, the second composition may comprise the first concentrations of the plurality of different biomolecules and no concentration of the one or more analytes. In some embodiments, the one or more second biomolecule coronas and the supernatant of the second composition may be obtained by contacting the second composition with a second surface. In some embodiments, the first surface and the second surface may have the same physicochemical properties. In some embodiments, the first concentration and the second concentration may be different. In some embodiments, the physicochemical properties may be composition, core material, shell material, porosity size, surface charge, hydrophobicity, hydrophilicity, surface functionality, surface topography, surface curvature, shape, or any combination thereof. In other embodiments, in (c), the first concentration an the second concentration are different.

The order in which components are combined to form the first composition and the second composition is not particularly limited. In some embodiments, a biomolecule may be introduced to a mixture comprising a surface prior to being introduced to an analyte. In some embodiments, an analyte may introduced to a mixture comprising a surface before biomolecules are introduced into the mixture. In some embodiments, a biomolecule may be introduced to a surface first and incubated to form a protein corona, and then the protein corona may be exposed to a solution comprising an analyte to form a first composition that is also incubated with the surface. For example, a plasma sample may be incubated for 1 hour at 37 degrees C. with magnetic nanoparticles to form a protein corona on the nanoparticles, and then the supernatant is removed while immobilizing the magnetic particles using a magnetic field. The nanoparticles with the protein corona may then be suspended in a buffer containing the analyte and incubated for 5 minutes at room temperature, which can result in changes to the protein corona due to interactions with the analyte. The supernatant may again be removed and the nanoparticles washed before further processing to assay proteins in the protein corona.

The order of obtaining the first assay data and second assay data is not particularly limited, and may be performed various sequences. In some embodiments, (c) providing a second assay data may occur prior to (b) assaying the one or more first biomolecule coronas and/or a supernatant of the first composition and subsequent to (a) contacting a surface with a first composition comprising first concentrations of a plurality of different biomolecules and a first concentration of one or more analytes to form one or more biomolecule coronas. In some embodiments, (c) providing a second assay data may occur prior to (b) assaying the one or more first biomolecule coronas and/or a supernatant of the first composition and prior to (a) contacting a surface with a first composition comprising first concentrations of a plurality of different biomolecules and a first concentration of one or more analytes to form one or more biomolecule coronas.

In one aspect, disclosed herein is a method of identifying biomolecule-analyte interactions in a subject comprising: (a) contacting a first surface with a first composition comprising concentrations of a plurality of different biomolecules and a first concentration of one or more analytes to form one or more first biomolecule corona, wherein the plurality of different biomolecules are derived from a biological sample of the subject; (b) assaying the one or more first biomolecule coronas and/or a supernatant of the first composition to obtain a first assay data representing quantities of the biomolecules in the first biomolecule coronas and/or the supernatant of the first composition; (c) contacting a second surface with a second composition comprising concentrations of a plurality of different biomolecules and a second concentration of one or more analytes to form one or more second biomolecule corona, wherein the plurality of different biomolecules are derived from the biological sample of the subject; (d) assaying the one or more second biomolecule coronas and/or a supernatant of the second composition to obtain a second assay data representing quantities of the biomolecules in the second biomolecule coronas and/or the supernatant of the second composition; and (e) identifying biomolecules that interact with the analytes based on variations between the first assay data and second assay data. In such methods, a variation may indicate responsiveness, for example, to a drug, and this may aid to inform dosing protocol based on pharmokinetics. In some embodiments, an interaction between a biomolecule and analyte may be identified through applying statistical analysis. In some embodiments, an interaction between a biomolecule and analyte may be identified through detecting statistically significant deviations. For example, analysis using the Student's t-test may be used to identify statistically significant variations in the assay data that can be associated with the analyte. This may be, in some embodiments, if the relative abundance measured in the corona or supernatant changes by for example 1.2×, 1.5×, 2×, 4×, 10×, 100× significantly (p-value<than 5%).

In some embodiments, the first concentration and the second concentration may be at least 1 parts per billion (ppb), 5 ppb, 10 ppb, 50 ppb, 100 ppb, 200 ppb, 300 ppb, 400 ppb, 500 ppb, 600 ppb, 700 ppb, 800 ppb, 900 ppb, 1 parts per million (ppm), 5 ppm, 10 ppm, 50 ppm, 100 ppm, 200 ppm, 300 ppm, 400 ppm, 500 ppm, 600 ppm, 700 ppm, 800 ppm, 900 ppm, 1000 ppm, 1500 ppm, 2000 ppm, 2500 ppm, 3000 ppm, 4000 ppm, 5000 ppm, 10000 ppm, 100000 ppm, or more. In other embodiments, the second concentration may be 0 ppb. In some embodiments, the first concentration of the one or more analytes is different from the second concentration of the one or more analytes.

In some embodiments, the first assay data may be acquired experimentally. In some embodiments, the second assay data may be retrieved from a database or reference source. In some embodiments, the second assay data may be derived from a database, reference source, or empirical data. In some embodiments, the second assay data may be acquired experimentally.

In some embodiments, (c) is performed prior to (b). In other embodiments, (c) is performed prior to (a). In yet other embodiments, (c) and (a) are performed, at least in part, during the same time.

In some embodiments, a biological sample derived from a subject may comprise plasma. In some embodiments, a biological sample is collected from a subject. In some embodiments, the method may comprise co-incubating a plurality of biological samples derived from a subject with different concentrations of an analyte to examine how a subject may respond to a drug (e.g., response, effectiveness, side effects) and/or identify difference in an interactome. In some embodiments, a biological sample derived from a subject may be a substantially cell-free sample (e.g., plasma or serum). In some embodiments, a biological sample is derived from a cell culture. For example, the biological sample may be derived from cell culture media.

In some embodiments, the method may comprise providing a plurality of samples comprising the biomolecules. In some embodiments, the method may comprise splitting a sample comprising biomolecules into two or more partitions and contacting and assaying with two or more concentrations of analyte. In some embodiments, the method may comprise splitting a sample comprising biomolecules into three or more partitions and contacting and assaying with three or more concentrations of analyte.

In some examples, the method may comprise simultaneously testing a plurality of analytes against a sample (comprising biomolecules and surfaces). For example, 50 different analytes may be tested simultaneously where a batch of a biological sample may be split into 5 partitions and each partition may be contacted with 10 different analytes; any partition in which a change in composition (e.g., of the corona or supernatant) is detected may be further investigated to narrow down and identify the biomolecule-analyte interaction. In some embodiments, the method includes simultaneously testing at least 10, at least 20, at least 50, or at least 100 analytes.

In some embodiments, the method may comprise contacting a plurality (e.g., two or more) of surfaces with an analyte, where each surface differs from another by at least one physicochemical property. In some embodiments, each surface may differ by at least composition, size, surface charge, hydrophobicity, hydrophilicity, surface functionality, surface topogragraphy, surface curvature, shape, or any combination thereof. In some embodiments, at least 2, at least 3, at least 4, at least 5, or at least 10 surfaces with different physicochemical properties are contacted with the analyte and biomolecules.

In some embodiments, the method may comprise contacting a plurality (e.g., two or more) of analytes with a biomolecule on a biomolecule corona, where each analyte is different. In some embodiments, each analyte of the plurality may form a complex with a biomolecule coupled to a surface. In some embodiments, each analyte may form a complex with a different biomolecule coupled to a surface.

In some embodiments, contacting may comprise incubating a surface, a biomolecule, an analyte, or a combination thereof. In some embodiments, contacting may comprise incubating the analyte with a biomolecule corona (e.g., biomolecules adsorbed onto a surface). In some embodiments, incubating is performed at a temperature from about 0° C. to about 120° C. In some embodiments, incubating is performed at a temperature from about 0° C. to about 100° C. In some embodiments, incubating is performed at a temperature from about 0° C. to about 90° C. In some embodiments, incubating is performed at a temperature from about 0° C. to about 80° C. In some embodiments, incubating is performed at a temperature from about 10° C. to about 70° C. In some embodiments, incubating is performed at a temperature from about 15° C. to about 50° C. In some embodiments, incubating is performed at a temperature from about 20° C. to about 45° C. In some embodiments, incubating occurs for a length of time of about at least about 15 seconds. In some embodiments, incubating occurs for a length of time of about at least about 30 seconds. In some embodiments, incubating occurs for a length of time of about at least about 1 minute. In some embodiments, incubating occurs for a length of time of about at least about 5 minutes. In some embodiments, incubating occurs for a length of time of about at least about 15 minutes. In some embodiments, incubating occurs for a length of time of about at least about 30 minutes. In some embodiments, incubating occurs for a length of time of about 5 minutes to about 24 hours. In some embodiments, incubating occurs for a length of time of about 5 minutes to about 12 hours. In some embodiments, incubating occurs for a length of time of about 15 minutes to about 12 hours. In some embodiments, incubating occurs for a length of time of about 30 minutes to about 12 hours. In some embodiments, incubating occurs for a length of time of about 30 seconds to about 12 hours.

In some embodiments, contacting may comprise adjusting a pH of a mixture (e.g. a solution) comprising a biomolecule corona. In some embodiments, the mixture may comprise a surface, a biomolecule, an analyte, or any combination thereof. In some embodiments, a pH may be adjusted for a mixture comprising a biomolecule corona (e.g., biomolecule adsorbed onto a surface) and an analyte. A pH may be adjusted such that a component of a biomolecule (e.g., a protein), for example, is inhibited from forming an interaction between the biomolecule and an analyte.

In some embodiments, assaying may comprise denaturing and/or chemically treating components of a mixture. In some embodiments, denaturing and/or chemical treatment may comprise heating, reducing (e.g., chemical reduction), alkylation, digestion (e.g., protease digestion), or a combination thereof.

In some embodiments, assaying and identifying may comprise using workflows (MS analysis of biomolecule corona composition and/or supernatant) as described in WO2020/096631 and WO2021/026172, both of which are incorporated herein by reference in their entirety. Feature intensities refer to the intensity of a discrete spike (“feature”) seen on a plot of mass to charge ratio versus intensity from a mass spectrometry run of a sample. These features can correspond to variably ionized fragments of biomolecules (e.g., peptides and/or proteins). Using the data analysis methods described in WO2020/096631, feature intensities may be sorted into protein groups, for example. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence.

In some embodiments, in assaying may comprise identifying proteins or protein groups. In some embodiments, assaying may be capable of identifying 1 to about 50,000 protein groups. In some embodiments, assaying may be capable of identifying 1 to about 40,000 protein groups. In some embodiments, assaying may be capable of identifying 1 to about 30,000 protein groups. In some embodiments, assaying may be capable of identifying 1 to about 20,000 protein groups. In some embodiments, assaying may be capable of identifying 1 to about 10,000 protein groups. In some embodiments, assaying may be capable of identifying 1 to about 5,000 protein groups. In some embodiments, assaying may be capable of identifying 1 to about 1,000 protein groups. In some embodiments, assaying may be capable of identifying 1 to about 500 protein groups. In some embodiments, both the first assay data and second assay data identify at least 100 protein groups. In some embodiments, both the first assay data and second assay data identify at least 200 protein groups. In some embodiments, both the first assay data and second assay data identify at least 500 protein groups. In some embodiments, both the first assay data and second assay data identify at least 1000 protein groups. Assaying may comprise centrifuging a supernatant comprising a complex, where the complex comprises a biomolecule-analyte interaction. In some embodiments, assaying may comprise quantifying at least one one biomolecule corona or a biomolecule-analyte interaction in a sample through Mass Spectrometry, such as liquid chromatography tandem mass spectrometry. In some embodiments, the mass spectrometry may be a targeted mass spectrometry, such as multiple reaction monitoring (MRM/SRM) or parallel reaction monitoring (PRM). For example, the targeted detection may include proteins associated with a particular diseased state. The targeted detection may improve quantification for the proteins of interest.

In some embodiments, assaying may comprise high throughput single molecule protein sequencing. For example, peptides may be sequenced using TIME DOMAIN SEQUENCING from Quantum-SI.

In some embodiments, the method may further comprise, prior to (b) and subsequent to (a) isolating a biomolecule corona and/or a supernatant of the composition. In some embodiments, isolating may comprise magnetically isolating a biomolecule corona.

In some embodiments, the proteins within the protein corona may be digested using an ezyme. In some embodiments, the digestion is performed on the protein corona while attached to the surface. The enzyme may be a protease, such as trypsin. In some embodiments, the protein corona is first digested with a less-selective enzyme, such as Proteinase K, and then subsequently digested with a selective enzyme, such as trypsin. Without being bound to any particular theory, the non-selective enzyme may only fragment portions of the protein that are exposed to water, while unexposed regions may remain intact. Differences in the fragmentation pattern can indicate analyte interactions that have impacted protein interactions. In some embodiments, detection or quantification of different peptide sequences can indicate analyte interactions.

In some embodiments, proteins within the protein corona may be cross-linked and cross-linked peptides or proteins are detected. Differences in the cross-linked peptides or proteins detected can indication analyte interactions that have impacted protein interactions. In some embodiments, proteins within the protein corona may be cross-linked using a mass spectrometry cleavable cross-linking agent and then cross-linked proteins or peptides may be detected using mass spectrometry. In some embodiments, proteins within the protein corona may be cross-linked using a mass spectrometry cleavable cross-linking agent and subsequently digested with a protease, such as trypsin. In some embodiments, proteins within the protein corona may be cross-linked using a mass spectrometry cleavable cross-linking agent, digested with a protease, and then analyzed using mass spectrometry. In some embodiments, detection or quantification of different cross-linked peptides can indicate analyte interaction.

In some embodiments, the method may further comprise steps comprising repeating, incubating, isolating, and assaying as described elsewhere herein. In some embodiments, incubating, the isolating, and the assaying may yield a percent quantile normalized coefficient (QNCV) of variation of 30% or less, as determined by comparing a mass spectrometry feature. In some embodiments, incubating, the isolating, and the assaying may yield a percent quantile normalized coefficient (QNCV) of variation of 20% or less, as determined by comparing a mass spectrometry feature.

In some embodiments, identifying may comprise detecting variations across one or more samples. In some embodiments, one or more samples may be associated with a first assay and a second assay. In some embodiments, variations may be detected through comparing a first assay data and a second assay data.

In some embodiments, detecting variations may comprise detecting a change in a composition of a supernatant (e.g., a first supernatant or after contacting a biomolecule and analyte) in comparison to an original composition of the supernatant (e.g., a second supernatant or without contacting a biomolecule and an analyte). In some embodiments, an original composition of the supernatant may comprise a surface with a composition comprising concentrations of a plurality of different biomolecules without, or prior to, contact with an analyte.

In some embodiments, detecting variations may comprise detecting a change in a composition of a biomolecule corona (e.g., a first biomolecule corona or after contacting a biomolecule and analyte) in comparison to an original composition of the biomolecule corona (e.g., a second biomolecule corona or without contacting a biomolecule and an analyte). In some embodiments, a composition of second biomolecule corona may comprise the plurality of different biomolecules without, or prior to, contact with an analyte. In some embodiments, subsequent to contacting, a biomolecule corona may be altered such that a biomolecule that is interacting with an analyte (e.g., biomolecule of the biomolecule-analyte interaction) is absent from a biomolecule corona.

In some embodiments, detecting variations may comprise detecting a change in a composition of a supernatant in comparison to an original comparison of the supernant and may additionally comprise detecting a change in a composition of a biomolecule corona in comparison to an original composition of the biomolecule corona. In some embodiments, an original composition of the supernatant may comprise a surface with a composition comprising concentrations of a plurality of different biomolecules without, or prior to, contact with an analyte. In some embodiments, a composition of second biomolecule corona may comprise the plurality of different biomolecules without, or prior to, contact with an analyte. In some embodiments, subsequent to contacting, a biomolecule corona may be altered such that a biomolecule that is interacting with an analyte (e.g., biomolecule of the biomolecule-analyte interaction) is absent from a biomolecule corona.

In some embodiments, an abundance of the biomolecule-analyte complex (e.g., interaction) may be measured by analyzing a composition of the biomolecule corona before and after contacting with the analyte, by analyzing a composition of a supernatant before and before contacting with the analyte, or both. In some embodiments, multiple measurements may be completed over time to result in information on dynamics of a biomolecule-analyte interaction relating to complex formation, complex dissociation, and potential direct or indirect molecular interactions.

In some embodiments, the method may be completed using multiple concentrations of analyte in a medium (e.g., biofluid or solvent). For example, the method may be completed using a first concentration of analyte and repeated one or more times using a different concentration of analyte. In such cases, a binding constant for the biomolecule-analyte interaction may be measured or determined.

In some embodiments, identifying a biomolecule-analyte interaction may be used for disease diagnosis or screening. In some embodiments, identifying a biomolecule-analyte interaction may comprise identifying a biological state. In some examples, a biological state may be a healthy biological state or a disease biological state. In some examples, a biological state may be a pharmacokinetic characteristic of a subject. In some examples, a biological state may be a risk of a disease state. In some embodiments, identifying may comprise identifying a signal transduction pathway, or a part thereof.

In some embodiments, physicochemical properties may comprise composition, core material, shell material, porosity size, surface charge, hydrophobicity, hydrophilicity, surface functionality, surface topogragraphy, surface curvature, shape, or any combination thereof.

Also provided herein are methods comprising incubating a biofluid with a surface and analyte to form a protein corona on the surface (e.g., such as a surface provided elsewhere herein). In some embodiments, the method comprises analyzing the proteins from the protein corona to obtain a protein corona signature associated with the biofluid and the analyte. In some embodiments, analyzing comprises analysis by mass spectrometry. In some embodiments, the method comprises comparing the protein corona to a suitable control protein corona signature to identify at least one protein-analyte interaction.

In some embodiments, the method comprises comparing the protein corona to a suitable control protein corona signature to identify at least one (e.g., at least 2, 3, 4, 5, 8, 10, or 15) protein-analyte interactions.

In some embodiments, the biofluid is a biofluid as described elsewhere herein. In some embodiments, the biofluid is plasma or serum. In some embodiments, the biofluid is plasma. In some embodiments, the biofluid is serum.

In some embodiments, incubating the biofluid with the surface and the analyte comprises incubating for at least 20 minutes. In some embodiments, incubating comprises incubating for at least 30 minutes (e.g., 40 minutes, 50 minutes, or one hour). In some embodiments, incubating comprises incubating for at most 24 hours (e.g., 16 hours, 12 hours, 8 hours, 4 hours, 3 hours, 1 hour, or 30 minutes). In some embodiments, incubating the biofluid with the surface and the analyte comprises incubating for any suitable amount of time as determined by one of skill in the art. In some embodiments, incubating the biofluid with the surface and the analyte comprises incubating at any suitable temperature. In some embodiments, incubating the biofluid with the surface and the analyte comprises incubating at a temperature of 15° C. to 75° C. In some embodiments, the incubating comprises incubating at at temperature of at least 10° C. (e.g., at least 15° C., 20° C., 30° C., 40° C., 50° C., 60° C., or 70° C.). In some embodiments, the incubating comprises incubating at a temperature of no greater than 100° C. (e.g., 90° C., 80° C., 70° C., 60° C., 50° C., 40° C., or 30° C.). In some embodiments, the incubating comprises incubating at a temperature of 15° C. to 50° C., 15° C.-30° C., 25° C.-50° C., 20° C.-40° C., or 25° C-75° C.

In some embodiments, the proteins from the protein corona are digested before analysis.

In some embodiments, the method further comprises sepatin the protein corona and surface from a supernatant before analyzing the protein corona.

### Surfaces

The surfaces that may be used with the disclosure of the present application include, but are not limited to, those disclosed WO2020/096631, which is hereby incorporated by reference in its entirety. In some aspects, a surface may be a particle or a bead. In some aspects, a surface may be a particle. In some embodiments, a particle may be a micelle, liposome, iron oxide particle, silver particle, gold particle, palladium particle, quantum dots, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle,, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, and 2-(3-aminopropylamino)ethanol particle, protein functionalized particle, ubiquitin functionalized particle, polysaccharide coated particle, or dextran functionalized particle. In some embodiments, a particle may comprise iron oxide.

In some embodiments, a particle may be a nanoparticle or a microparticle. In some embodiments, the particles disclosed herein can have a diameter of at least 10 nm, at least 100 nm, at least 200 nm, at least 300 nm, at least 400 nm, at least 500 nm, at least 600 nm, at least 700 nm, at least 800 nm, at least 900 nm, at least 1000 nm, at least 1100 nm, at least 1200 nm, at least 1300 nm, at least 1400 nm, at least 1500 nm, at least 1600 nm, at least 1700 nm, at least 1800 nm, at least 1900 nm, at least 2000 nm, at least 2100 nm, at least 2200 nm, at least 2300 nm, at least 2400 nm, at least 2500 nm, at least 2600 nm, at least 2700 nm, at least 2800 nm, at least 2900 nm, at least 3000 nm, at least 3100 nm, at least 3200 nm, at least 3300 nm, at least 3400 nm, at least 3500 nm, at least 3600 nm, at least 3700 nm, at least 3800 nm, at least 3900 nm, at least 4000 nm, at least 4100 nm, at least 4200 nm, at least 4300 nm, at least 4400 nm, at least 4500 nm, at least 4600 nm, at least 4700 nm, at least 4800 nm, at least 4900 nm, at least 5000 nm, at least 5100 nm, at least 5200 nm, at least 5300 nm, at least 5400 nm, at least 5500 nm, at least 5600 nm, at least 5700 nm, at least 5800 nm, at least 5900 nm, at least 6000 nm, at least 6100 nm, at least 6200 nm, at least 6300 nm, at least 6400 nm, at least 6500 nm, at least 6600 nm, at least 6700 nm, at least 6800 nm, at least 6900 nm, at least 7000 nm, at least 7100 nm, at least 7200 nm, at least 7300 nm, at least 7400 nm, at least 7500 nm, at least 7600 nm, at least 7700 nm, at least 7800 nm, at least 7900 nm, at least 8000 nm, at least 8100 nm, at least 8200 nm, at least 8300 nm, at least 8400 nm, at least 8500 nm, at least 8600 nm, at least 8700 nm, at least 8800 nm, at least 8900 nm, at least 9000 nm, at least 9100 nm, at least 9200 nm, at least 9300 nm, at least 9400 nm, at least 9500 nm, at least 9600 nm, at least 9700 nm, at least 9800 nm, at least 9900 nm, at least 10000 nm or from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm, from 10 to 100 nm, from 100 to 200 nm, from 200 to 300 nm, from 300 to 400 nm, from 400 to 500 nm, from 500 to 600 nm, from 600 to 700 nm, from 700 to 800 nm, from 800 to 900 nm, from 900 to 1000 nm, from 1000 to 1100 nm, from 1100 to 1200 nm, from 1200 to 1300 nm, from 1300 to 1400 nm, from 1400 to 1500 nm, from 1500 to 1600 nm, from 1600 to 1700 nm, from 1700 to 1800 nm, from 1800 to 1900 nm, from 1900 to 2000 nm, from 2000 to 2100 nm, from 2100 to 2200 nm, from 2200 to 2300 nm, from 2300 to 2400 nm, from 2400 to 2500 nm, from 2500 to 2600 nm, from 2600 to 2700 nm, from 2700 to 2800 nm, from 2800 to 2900 nm, from 2900 to 3000 nm, from 3000 to 3100 nm, from 3100 to 3200 nm, from 3200 to 3300 nm, from 3300 to 3400 nm, from 3400 to 3500 nm, from 3500 to 3600 nm, from 3600 to 3700 nm, from 3700 to 3800 nm, from 3800 to 3900 nm, from 3900 to 4000 nm, from 4000 to 4100 nm, from 4100 to 4200 nm, from 4200 to 4300 nm, from 4300 to 4400 nm, from 4400 to 4500 nm, from 4500 to 4600 nm, from 4600 to 4700 nm, from 4700 to 4800 nm, from 4800 to 4900 nm, from 4900 to 5000 nm, from 5000 to 5100 nm, from 5100 to 5200 nm, from 5200 to 5300 nm, from 5300 to 5400 nm, from 5400 to 5500 nm, from 5500 to 5600 nm, from 5600 to 5700 nm, from 5700 to 5800 nm, from 5800 to 5900 nm, from 5900 to 6000 nm, from 6000 to 6100 nm, from 6100 to 6200 nm, from 6200 to 6300 nm, from 6300 to 6400 nm, from 6400 to 6500 nm, from 6500 to 6600 nm, from 6600 to 6700 nm, from 6700 to 6800 nm, from 6800 to 6900 nm, from 6900 to 7000 nm, from 7000 to 7100 nm, from 7100 to 7200 nm, from 7200 to 7300 nm, from 7300 to 7400 nm, from 7400 to 7500 nm, from 7500 to 7600 nm, from 7600 to 7700 nm, from 7700 to 7800 nm, from 7800 to 7900 nm, from 7900 to 8000 nm, from 8000 to 8100 nm, from 8100 to 8200 nm, from 8200 to 8300 nm, from 8300 to 8400 nm, from 8400 to 8500 nm, from 8500 to 8600 nm, from 8600 to 8700 nm, from 8700 to 8800 nm, from 8800 to 8900 nm, from 8900 to 9000 nm, from 9000 to 9100 nm, from 9100 to 9200 nm, from 9200 to 9300 nm, from 9300 to 9400 nm, from 9400 to 9500 nm, from 9500 to 9600 nm, from 9600 to 9700 nm, from 9700 to 9800 nm, from 9800 to 9900 nm, from 9900 to 10000 nm. The diameter can be measured by dynamic light scattering (DLS) as an indirect measure of size. The DLS measurement can be an ‘intensity-weighted’ average, which means the size distribution that the mean is calculated from can be weighted by the sixth power of radius. This can be referred to herein as ‘z-average’ or ‘intensity-mean’.

Alternatively, in some embodiments, the particles disclosed herein can have a radius of at least 10 nm, at least 100 nm, at least 200 nm, at least 300 nm, at least 400 nm, at least 500 nm, at least 600 nm, at least 700 nm, at least 800 nm, at least 900 nm, at least 1000 nm, at least 1100 nm, at least 1200 nm, at least 1300 nm, at least 1400 nm, at least 1500 nm, at least 1600 nm, at least 1700 nm, at least 1800 nm, at least 1900 nm, at least 2000 nm, at least 2100 nm, at least 2200 nm, at least 2300 nm, at least 2400 nm, at least 2500 nm, at least 2600 nm, at least 2700 nm, at least 2800 nm, at least 2900 nm, at least 3000 nm, at least 3100 nm, at least 3200 nm, at least 3300 nm, at least 3400 nm, at least 3500 nm, at least 3600 nm, at least 3700 nm, at least 3800 nm, at least 3900 nm, at least 4000 nm, at least 4100 nm, at least 4200 nm, at least 4300 nm, at least 4400 nm, at least 4500 nm, at least 4600 nm, at least 4700 nm, at least 4800 nm, at least 4900 nm, at least 5000 nm, at least 5100 nm, at least 5200 nm, at least 5300 nm, at least 5400 nm, at least 5500 nm, at least 5600 nm, at least 5700 nm, at least 5800 nm, at least 5900 nm, at least 6000 nm, at least 6100 nm, at least 6200 nm, at least 6300 nm, at least 6400 nm, at least 6500 nm, at least 6600 nm, at least 6700 nm, at least 6800 nm, at least 6900 nm, at least 7000 nm, at least 7100 nm, at least 7200 nm, at least 7300 nm, at least 7400 nm, at least 7500 nm, at least 7600 nm, at least 7700 nm, at least 7800 nm, at least 7900 nm, at least 8000 nm, at least 8100 nm, at least 8200 nm, at least 8300 nm, at least 8400 nm, at least 8500 nm, at least 8600 nm, at least 8700 nm, at least 8800 nm, at least 8900 nm, at least 9000 nm, at least 9100 nm, at least 9200 nm, at least 9300 nm, at least 9400 nm, at least 9500 nm, at least 9600 nm, at least 9700 nm, at least 9800 nm, at least 9900 nm, at least 10000 nm or from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm, from 10 to 100 nm, from 100 to 200 nm, from 200 to 300 nm, from 300 to 400 nm, from 400 to 500 nm, from 500 to 600 nm, from 600 to 700 nm, from 700 to 800 nm, from 800 to 900 nm, from 900 to 1000 nm, from 1000 to 1100 nm, from 1100 to 1200 nm, from 1200 to 1300 nm, from 1300 to 1400 nm, from 1400 to 1500 nm, from 1500 to 1600 nm, from 1600 to 1700 nm, from 1700 to 1800 nm, from 1800 to 1900 nm, from 1900 to 2000 nm, from 2000 to 2100 nm, from 2100 to 2200 nm, from 2200 to 2300 nm, from 2300 to 2400 nm, from 2400 to 2500 nm, from 2500 to 2600 nm, from 2600 to 2700 nm, from 2700 to 2800 nm, from 2800 to 2900 nm, from 2900 to 3000 nm, from 3000 to 3100 nm, from 3100 to 3200 nm, from 3200 to 3300 nm, from 3300 to 3400 nm, from 3400 to 3500 nm, from 3500 to 3600 nm, from 3600 to 3700 nm, from 3700 to 3800 nm, from 3800 to 3900 nm, from 3900 to 4000 nm, from 4000 to 4100 nm, from 4100 to 4200 nm, from 4200 to 4300 nm, from 4300 to 4400 nm, from 4400 to 4500 nm, from 4500 to 4600 nm, from 4600 to 4700 nm, from 4700 to 4800 nm, from 4800 to 4900 nm, from 4900 to 5000 nm, from 5000 to 5100 nm, from 5100 to 5200 nm, from 5200 to 5300 nm, from 5300 to 5400 nm, from 5400 to 5500 nm, from 5500 to 5600 nm, from 5600 to 5700 nm, from 5700 to 5800 nm, from 5800 to 5900 nm, from 5900 to 6000 nm, from 6000 to 6100 nm, from 6100 to 6200 nm, from 6200 to 6300 nm, from 6300 to 6400 nm, from 6400 to 6500 nm, from 6500 to 6600 nm, from 6600 to 6700 nm, from 6700 to 6800 nm, from 6800 to 6900 nm, from 6900 to 7000 nm, from 7000 to 7100 nm, from 7100 to 7200 nm, from 7200 to 7300 nm, from 7300 to 7400 nm, from 7400 to 7500 nm, from 7500 to 7600 nm, from 7600 to 7700 nm, from 7700 to 7800 nm, from 7800 to 7900 nm, from 7900 to 8000 nm, from 8000 to 8100 nm, from 8100 to 8200 nm, from 8200 to 8300 nm, from 8300 to 8400 nm, from 8400 to 8500 nm, from 8500 to 8600 nm, from 8600 to 8700 nm, from 8700 to 8800 nm, from 8800 to 8900 nm, from 8900 to 9000 nm, from 9000 to 9100 nm, from 9100 to 9200 nm, from 9200 to 9300 nm, from 9300 to 9400 nm, from 9400 to 9500 nm, from 9500 to 9600 nm, from 9600 to 9700 nm, from 9700 to 9800 nm, from 9800 to 9900 nm, from 9900 to 10000 nm.

In some embodiments, the particles disclosed herein have a diameter of 100 nm to 400 nm. In other examples, the particles disclosed herein have a radius of 100 nm to 400 nm. Particle size can be determined by a number of techniques, such as dynamic light scattering or electron microscopy (e.g., SEM, TEM). Particles disclosed herein can be nanoparticles or microparticles.

In some aspects, particles may have a homogenous size distribution or a heterogeneous size distribution. Polydispersity index (PDI), which can be measured by techniques such as dynamic light scattering, is a measure of the size distribution. A low PDI indicates a more homogeneous size distribution and a higher PDI indicates a more heterogeneous size distribution. For example, particles disclosed herein can have a PDI of less than 0.5, less than 0.4, less than 0.3, less than 0.2, less than 0.15, or less than 0.1. In particular embodiments, the particles (e.g., surfaces) disclosed herein have a PDI of less than 0.1.

In some embodiments, particles disclosed herein can have a range of different surface charges. Particles can be negatively charged, positively charged, or neutral in charge. In some embodiments, particles have a surface charge of −500 mV to −450 mV, −450 mV to −400 mV, −400 mV to −350 mV, −350 mV to −300 mV, −300 mV to −250 mV, −250 mV to −200 mV, −200 mV to −150 mV, −150 mV to −100 mV, −100 mV to −90 mV, −90 mV to −80 mV, −80 mV to −70 mV, −70 mV to −60 mV, −60 mV to −50 mV, −50 mV to −40 mV, −40 mV to −30 mV, −30 mV to −20 mV, −20 mV to −10 mV, −10 mV to 0 mV, 0 mV to 10 mV, 10 mV to 20 mV, 20 mV to 30 mV, 30 mV to 40 mV, 40 mV to 50 mV, 50 mV to 60 mV, 60 mV to 70 mV, 70 mV to 80 mV, 80 mV to 90 mV, 90 mV to 100 mV, 100 mV to 110 mV, 110 mV to 120 mV, 120 mV to 130 mV, 130 mV to 140 mV, 140 mV to 150 mV, 150 mV to 200 mV, 200 mV to 250 mV, 250 mV to 300 mV, 300 mV to 350 mV, 350 mV to 400 mV, 400 mV to 450 mV, 450 mV to 500 mV, −500 mV to −400 mV, −400 mV to −300 mV, −300 mV to −200 mV, −200 mV to −100 mV, −100 mV to 0 mV, 0 mV to 100 mV, 100 mV to 200 mV, 200 mV to 300 mV, 300 mV to 400 mV, or 400 mV to 500 mV. In particular examples, particles disclosed herein have a surface charge of −60 mV to 60 mV. The surface charge may be evaluated by zeta potential analysis.

In some embodiments, the one or more surfaces comprises a first distinct surface and a second distinct surface, wherein the first distinct surface and the second distinct surface comprise a carboxylate material, wherein the first distinct, the second distinct surface, or both are a nanoparticle. In some embodiments, the one or more surfaces comprises a first distinct surface and a second distinct surface, wherein the first distinct surface and the second distinct surface comprise a surface charge of from 0 mV and −50 mV, wherein the first distinct surface, the second distinct surface, or both have a diameter of less than 400 nm. In some embodiments, the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a negative surface charge.

In some embodiments, the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than 20 mV. In some embodiments, the one or more surfaces comprises a first distinct surface and a second distinct surface, wherein the first distinct surface and the second distinct surface are microparticles, wherein the first distinct surface has a negative surface charge, and wherein the second distinct surface has a positive surface charge. In some embodiments, the the one or more surfaces comprises a subset of negatively charged nanoparticles, wherein each surface of the subset differ by at least one surface chemical group. In some embodiments, the one or more surfaces comprises a first distinct surface, a second distinct surface, and a third distinct surface, wherein the first distinct surface, the second distinct surface, and the third distinct surface comprise iron oxide cores, and are less than about 500 nm in diameter, and wherein the first distinct surface comprises a negative charge of less −40 mV, the second distinct surface comprises a positive charge of more than 20 mV, and the third distinct surface comprises a negative charge of −20 m V to −40 mV.

In some embodiments, the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different. As a non-limiting example, the distinct surfaces may both be particles having the same diameter and a polymeric surface, but the surface charges and hydrophobicity of the particles are different. In some embodiments, the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.

In some embodiments, the method may comprise utilizing one or more surfaces. In such embodiments, a first surface and a second surface may be the same surface and not differ in a physicochemical property. In some cases, a first surface and a second surface may share at least two physicochemical properties and differ by at least one physicochemical property, such that the first surface and the second surface are different. A physicochemical property may comprise size, charge, core material, shell material, porosity, or surface hydrophobicity. In further embodiments, size may refer to diameter or radius, as measured by dynamic light scattering, SEM, TEM, or any combination thereof.

In some embodiments, a surface may comprise a nanoparticle. In some embodiments, the surface may comprise a microparticle. For example, a sample may comprise a plurality of the same surface, where one or more surfaces among the plurality of surfaces may differ in size or charge. In some embodiments, a surface may be magnetic. In some embodiments, a surface may comprise a superparamagnetic iron oxide particle. In some embodiments, a surface may comprise an iron oxide material. In some embodiments, a surface may comprise an iron oxide core. In some embodiments, a surface may comprise iron oxide crystals embedded in a polystyrene core.

In some embodiments, each distinct surface of the two or more distinct magnetic surfaces is a superparamagnetic iron oxide particle. In some embodiments, each distinct surface of the two or more distinct magnetic surfaces comprise an iron oxide core. In some embodiments, each one distinct surface of the two or more distinct magnetic surfaces has iron oxide crystals embedded in a polystyrene core. In some embodiments, at least one surface of the two or more distinct magnetic surfaces comprises a polymer coating.

In some embodiments, a surface may comprise a polymer coating. In some embodiments, a surface may comprise a carboxylated polymer, an aminated polymer, a zwitterionic polymer, or any combination thereof. In some embodiments, a surface may comprise an iron oxide core with a silica shell coating. In some embodiments, a surface may comprise an iron oxide core with a poly(N-(3-(dimethylamino)propyl) methacrylamide) (PDMAPMA) coating. In some embodiments, a surface may comprise a carboxyl-containing outer surface. In some embodiments, a surface may comprise an amine-containing outer surface. In some embodiments, a surface may comprise an iron oxide core with a poly(oligo(ethylene glycol) methyl ether methacrylate) (POEGMA) coating.

In some embodiments, a surface may comprise a negative surface charge. In some embodiments, a surface may comprise a positive surface charge. In some embodiments, a surface may comprise a neutral surface charge.

### Supernatants

In some aspects, a supernatant may refer to the surrounding environment of a biomolecule corona (e.g., biomolecules adsorbed onto a surface). A supernatant may comprise surfaces, unbound biomolecules, unbound analyte, analytes interacting with unbound biomolecules, or a combination thereof. In some embodiments, the composition of the supernatant is dynamic and will change as parameters are changed or complexes are formed. In some embodiments, the supernatant is a fluid separated from the surfaces after formation of the biomolecule corona. In some embodiments, the supernatant is a fluid separated from the surfaces after formation of the biomolecule corona step (a) in the methods disclosed herein. In some embodiments, the dynamic range of biomolecules in the supernatant is at least five orders of magnitude greater that biomolecules in the biomolecule corona.

As a non-limiting example, a surface, such as magnetic nanoparticles, may be incubated with an analyte and biological sample to form a biomolecule corona on the surface. The supernatant may be obtained by isolating the surrounding fluid containing any unbound biomolecules and unbound analyte. The isolating could be performed, in some embodiments, by removing the surrounding fluid while a magnetic field is applied to immobilize the nanoparticle and biomolecule corona. In some embodiments, the isolating may be performed by magnetic separation, size exclusion (e.g., filtration), by binding free proteins to other surfaces, by crosslinking the NPs and bound biomolecules to aidseperation, and the like.

### Biomolecules

In some embodiments, a biological sample may comprise a biomolecule. In some embodiments, biomolecules may be interacting with a surface, or be unbound in a supernatant. In some embodiments, biomolecules may interact with an analyte and form a biomolecule-molecule complex.

In some embodiments, a composition may comprise a plurality of biomolecules. In some embodiments, a plurality of biomolecules may be different biomolecules. In some embodiments, a biomolecule may be a nucleic acid, a small molecule, a protein, a lipid, antibody, a polysaccharide, or a metabolite thereof. In some embodiments, a biomolecule may be a protein.

In some embodiments, a protein may be a shuttle protein, a ligand, a member of a protein complex, comprise a low complexity region, comprise an interaction domain, involved in signal transduction, or any combination thereof. In some embodiments, a protein may be a shuttle protein. In some embodiments, a protein may be a ligand. In some embodiments, a protein may be a member of a protein complex. In some embodiments, a protein may comprise a low complexity region. In some embodiments, a protein may comprise an interaction domain. In some embodiments, a protein may be involved in signal transduction.

In some embodiments, a biomolecule may be adhered to a surface. In some embodiments, a biomolecule may be adsorbed onto a surface. In some embodiments, a biomolecule may reversibly interact with a surface.

### Analytes

In some embodiments, an analyte may be provided in a biological sample (e.g., a biofluid). In some embodiments, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, an analyte may not be provided in a biological sample (e.g., a biofluid). In such cases, an analyte may be provided in a a liquid or gaseous medium. In some cases, an analyte may be provided in a solvent (e. g, water, alcohol, a polar solvent, a nonpolar solvent). In some cases, the solvent may be sufficient or configured to dissolve the analyte (e.g., through adjusting a temperature, a pH, or by adding one or more additional components to enhance solubility).

In some embodiments, the concentration of an analyte in the composition (e.g., the first composition in the methods described above) may be at least 1 parts per billion (ppb), 5 ppb, 10 ppb, 50 ppb, 100 ppb, 200 ppb, 300 ppb, 400 ppb, 500 ppb, 600 ppb, 700 ppb, 800 ppb, 900 ppb, 1 parts per million (ppm), 5 ppm, 10 ppm, 50 ppm, 100 ppm, 200 ppm, 300 ppm, 400 ppm, 500 ppm, 600 ppm, 700 ppm, 800 ppm, 900 ppm, 1000 ppm, 1500 ppm, 2000 ppm, 2500 ppm, 3000 ppm, 4000 ppm, 5000 ppm, 10000 ppm, 100000 ppm, or more.

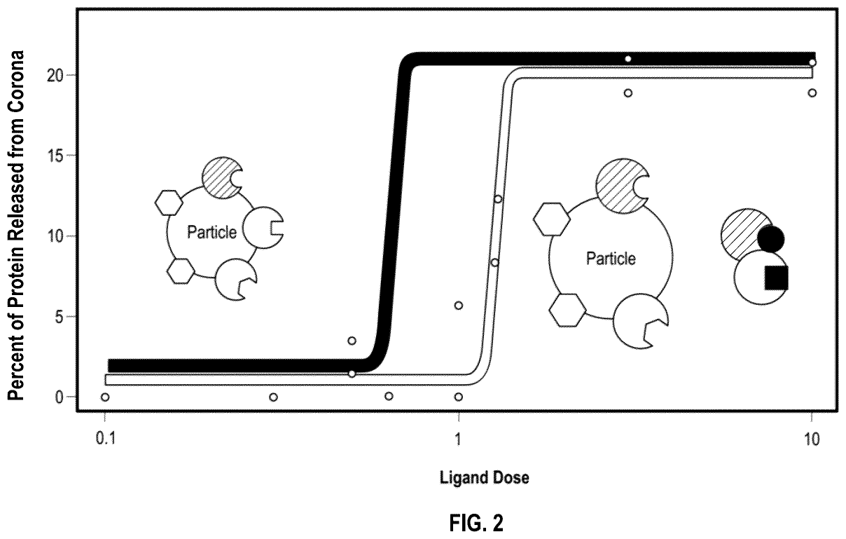

In some embodiments, the concentration of an analyte may influence the percentage of a biomolecule that is bound in a biomolecule corona. In some embodiments, a relationship between the concentration of an analyte and percentage of a bound biomolecule is direct. For example, a higher concentration of analyte may result in a lower percentage of certain biomolecules bound in a corona (e.g., decoupled from a surface). With reference to FIG. 2 as an example, an increase in ligand dose reduces the percentage of a biomolecule (e.g., protein) bound in a corona, until a maximum amount is reached. As another example, the amount (e.g., percentage) of a biomolecule in the corona may increase with the concentration of the analyte.

In some embodiments, an analyte may be a drug, a therapeutic agent, a nutrient, a biomolecule, a pathogen, or a metabolite thereof. In some embodiments, the analyte may be a drug. In some embodiments, a drug may comprise an antibody, a biotherapeutic, or a chemotherapeutic biomolecule. In some embodiments, a drug may comprise a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, an anti-hypertensive, or any combination thereof. In some embodiments, an analyte may be a pathogen. In some embodiments, a pathogen may comprise a bacteria, a virus, a fungus, a protozoa, or a worm. In some embodiments, a pathogen may comprise a hepatitis B virus, a hepatitis C virus, a COVID-19 virus, a Human Immunideficiency virus, or any other virus. In some embodiments, a therapeutic agent may comprise a recombinant preparation, a synthetic preparation, a natural preparation, or a combination thereof. In some embodiments, a therapeutic agent may comprise a cytokine or any pharmaceutically active ingredients. In some embodiments, a nutrient may comprise a fat, a protein, a carbohydrate, a mineral, a vitamin, an amino acid, or any other substance that provides nourishment essential for growth or maintenance of life. In some embodiments, the analyte is a non-naturally occurring small molecule. In some embodiments, the analyte is a non-naturally occurring small molecule having a molecular weigh less than 100 kD. In some embodiments, the analyte is a non-naturally occurring small molecule having a molecular weigh less than 50 kD. In some embodiments, the analyte is a non-naturally occurring small molecule having a molecular weigh less than 25 kD. In some embodiments, the analyte is a non-naturally occurring small molecule having a molecular weigh less than 10 kD. In some embodiments, the molecule comprises a drug. In some embodiments, the first composition comprises a biological sample, and the analyte is not present in the biological sample. In some embodiments, the first composition comprises a biological sample, the analyte is present in the biological sample, and additional analyte is added to the first composition.

In some embodiments, an analyte may be a biomolecule as described elsewhere herein. In such embodiments, a biomolecule may be a protein or a portion thereof. In some cases, a protein (or portion thereof) may comprise a receptor or enzyme configured to bind to the biomolecule adsorbed onto a surface (e.g., particle). In some embodiments, a protein may comprise a peptide sequence having a minimum length 3 amino acid (AA) residues, 4 AA residues, 5 AA residues, 6 AA residues, 7 AA residues, 8 AA residues, 9 AA residues, 10 AA residues, 15 AA residues, 20 AA residues, or more. In some embodiments, a protein may comprise a peptide sequence having a minimum length 7 amino acid (AA) residues. In some embodiments, an analyte may comprise a lipid. In some embodiments, an analyte may comprise a metabolite of a biomolecule.

In some embodiments, an analyte may be configured to interact with one or more different biomolecules (e.g., a protein). In some embodiments, one or more different biomolecules may comprise a protein. In some cases, an analyte may interact with an allosteric site on a protein. In some cases, an analyte may interact with an active site on a protein. In some cases, an analyte may interact with a target receptor site on a protein.

In some embodiments, an analyte may comprise a plurality of binding moieties. In some embodiments, an analyte may comprise two or more binding moieties. In some cases, a first binding moiety of two or more binding moieties may form a complex with a first biomolecule and a second binding moeitiy of two or more binding moieties may form a complex with a second biomolecule, where the first biomolecule and the second biomolecule are different biomolecules (e.g., differ by at least one physicochemical property). In such cases, each analyte molecule may interact with only one biomolecule. In some embodiments, assaying may detect all direct and interactions between a biomolecule and an analyte.

In some embodiments, a first binding moiety of two or more binding moieties may form a first complex with a first component of a biomolecule adsorbed to a surface and a second binding moieity of two or more binding moieties may form a second complex with a second component of the biomolecule adsorbed to a surface. In such cases, two or more analyte molecules may interact with a single biomolecule at different sites on the biomolecule (e. g, where the first component and the second component are different). In such cases, a first complex and second complex may be reach equilibrium or be in dynamic equilibrium. In some embodiments, an analyte may form a complex with only one biomolecule. In some embodiments, an analyte comprising two or more binding moieties may simultaneously form a complex with a first biomolecule and second biomolecule, where the first biomolecule and the second biomolecule are the same or different.

### Biomolecule Coronas

In some aspects, a biomolecule corona may comprise a surface with one or more different biomolecules adsorbed onto the surface. In some aspects, the biomolecule corona may further comprise an analyte interacting with a biomolecule of the biomolecule corona.

In some aspects, the one or more first biomolecule coronas comprise one or more different biomolecules. In some embodiments, the one or more first biomolecule coronas comprise the one or more analytes.

In some embodiments, a surface or a particle of the disclosure may be incubated with any biological sample disclosed herein to form a protein (or biomolecule) corona comprising at least 40 proteins or protein groups, at least 60 proteins or protein groups, at least 80 proteins or protein groups, at least 100 proteins or protein groups, at least 120 proteins or protein groups, at least 140 proteins or protein groups, at least 160 proteins or protein groups, at least 180 proteins or protein groups, at least 200 proteins or protein groups, at least 220 proteins or protein groups, at least 240 proteins or protein groups, at least 260 proteins or protein groups, at least 280 proteins or protein groups, at least 300 proteins or protein groups, at least 320 proteins or protein groups, at least 340 proteins or protein groups, at least 360 proteins or protein groups, at least 380 proteins or protein groups, at least 400 proteins or protein groups, at least 420 proteins or protein groups, at least 440 proteins or protein groups, at least 460 proteins or protein groups, at least 480 proteins or protein groups, at least 500 proteins or protein groups, at least 520 proteins or protein groups, at least 540 proteins or protein groups, at least 560 proteins or protein groups, at least 580 proteins or protein groups, at least 600 proteins or protein groups, at least 620 proteins or protein groups, at least 640 proteins or protein groups, at least 660 proteins or protein groups, at least 680 proteins or protein groups, at least 700 proteins or protein groups, at least 720 proteins or protein groups, at least 740 proteins or protein groups, at least 760 proteins or protein groups, at least 780 proteins or protein groups, at least 800 proteins or protein groups, at least 820 proteins or protein groups, at least 840 proteins or protein groups, at least 860 proteins or protein groups, at least 880 proteins or protein groups, at least 900 proteins or protein groups, at least 920 proteins or protein groups, at least 940 proteins or protein groups, at least 960 proteins or protein groups, at least 980 proteins or protein groups, at least 1000 proteins or protein groups, from 100 to 1000 proteins or protein groups, from 150 to 950 proteins or protein groups, from 200 to 900 proteins or protein groups, from 250 to 850 proteins or protein groups, from 300 to 800 proteins or protein groups, from 350 to 750 proteins or protein groups, from 400 to 700 proteins or protein groups, from 450 to 650 proteins or protein groups, from 500 to 600 proteins or protein groups, from 200 to 250 proteins or protein groups, from 250 to 300 proteins or protein groups, from 300 to 350 proteins or protein groups, from 350 to 400 proteins or protein groups, from 400 to 450 proteins or protein groups, from 450 to 500 proteins or protein groups, from 500 to 550 proteins or protein groups, from 550 to 600 proteins or protein groups, from 600 to 650 proteins or protein groups, from 650 to 700 proteins or protein groups, from 700 to 750 proteins or protein groups, from 750 to 800 proteins or protein groups, from 800 to 850 proteins or protein groups, from 850 to 900 proteins or protein groups, from 900 to 950 proteins or protein groups, from 950 to 1000 proteins or protein groups. In some embodiments, a surface may be incubated with a biological sample to form a protein corona comprising at least about 50 to 500 proteins or protein groups. In some embodiments, the number of proteins or protein groups detected is based on a false detection rate of less than 1%.

In some embodiments, a biological sample may comprise proteins or protein groups. In some embodiments, a biological sample may comprise at least 10 protein or protein groups, at least 100 protein or protein groups, at least 200 protein or protein groups, at least 300 protein or protein groups, at least 400 protein or protein groups, at least 500 protein or protein groups, at least 600 protein or protein groups, at least 700 protein or protein groups, at least 800 protein or protein groups, at least 900 protein or protein groups, at least 1000 protein or protein groups, at least 1100 protein or protein groups, at least 1200 protein or protein groups, at least 1300 protein or protein groups, at least 1400 protein or protein groups, at least 1500 protein or protein groups, at least 1600 protein or protein groups, at least 1700 protein or protein groups, or more. In some embodiments, a biological sample may comprise at least 100 protein or protein groups. In some embodiments, a biological sample may comprise at least 500 protein or protein groups. In some embodiments, a biological sample may comprise at least 1000 protein or protein groups.

In some embodiments, a plurality of different biomolecules may adsorb onto a surface to form one or more biomolecule coronas. In some embodiments, one or more analytes may adsorb onto the surface to form the one or more first biomolecule coronas. A biomolecule may differ from another biomolecule based on one or more physical or chemical properties (e.g., structural features, bonds, functional groups, molecular weight). In some embodiments, a biomolecule corona (e.g., a first biomolecule corona or a second biomolecule corona as discussed above) may comprise a a first physicochemical property selected from the group consisting of a magnetic core, a polystyrene core, a metal core, a gold core, a metal oxide core, an iron oxide core, a polymeric core, and a silica core and a second physicochemical property selected from the group consisting of a carboxylated surface, an amino surface, a silica surface, a polymer surface, a phosphate sugar functionalized surface, a phenol functionalized surface, a citrate functionalized surface, a Jeffamine surface, and a silica silanol surface.

In some embodiments, an interaction between a biomolecule and a surface may result in a biomolecule corona where the biomolecule is adsorbed onto the surface. In some embodiments, adsorption of the biomolecule is dynamic, where the biomolecules may bind on and off of the surface. In some embodiments, changing the composition of solution (e.g., by increasing or decreasing a concentration of analyte) may shift the equilibrium of biomolecules adsorbed onto the surface and result in more or less biomolecules adsorbed onto the surface (in comparison to being in the supernatant). In some embodiments, the interaction of a biomolecule with a surface is reversible and the process in either direction maintains a native proteoform. In some embodiments, an original biomolecule corona can be restored after completing an assay, for example, by diluting (e.g., decreasing) the concentration of an analyte. In convential thermal shift assays, binding of a protein and an analyte is not reversible and often requires denaturing the protein (e.g., biomolecule) to achieve precipitation for analysis. In some embodiments, the biomolecule corona and/or supernatant may be assayed before and after changes to the compositions. For example, after incubation to the form the biomolecule corona without an analyte, a portion may be aliquoted and analyzed. The analyte may be added to the unanalyzed portion, incubated in the presence of the analyte, and then a second portion may be aliquoted and analyzed. This process can be repeated as desired.

In some embodiments, reversibility of a biomolecule and surface interaction may allow the system to be manipulated in such a way to understand complex formation (e.g., biomolecule-analyte complex) at various conditions (e.g., concentrations, temperatures, pH, etc) within a sample. On the other hand, with conventional thermal shift assays, testing different parameters (e.g., concentrations, analytes) require a fresh sample for each run as the protein is heated and denatured, and therefore lost.

### Biomolecule-Analyte Interactions

In some embodiments, an interaction between a biomolecule and an analyte may comprise a coulombic interaction, a hydrogen bond, a Van der Waals interaction, solvent exclusion, hydrophobic effect, or a combination thereof.

In some embodiments, a biomolecule-analyte interaction is direct. In some embodiments, an biomolecule-analyte interaction is indirect. In some embodiments, a biomolecule-analyte interaction may comprise an electrostatic interaction between a biomolecule and an analyte. In some embodiments, an electrostatic interaction may comprise an ionic bond between a biomolecule and an analyte. In some embodiments, an electrostatic interaction may comprise a hydrogen bond between a biomolecule and an analyte. In some embodiments, an electrostatic interaction may comprise a Van der Waals interaction between a biomolecule and an analyte. In some embodiments, the electrostatic interaction may be reversible.

In some aspects, a biomolecule-analyte interaction may comprise a biomolecule-analyte complex. In some embodiments, a thermodynamic stability of the biomolecule-analyte complex may be greater than a thermodynamic stability of an interaction comprising a biomolecule-analyte interaction, where the biomolecule is further interacting with a surface. In such embodiments, a biomolecule-analyte complex may decouple from a surface, thereby altering a composition of the biomolecule corona. In some embodiments, decoupling may occur spontaneously. In some embodiments, decoupling may occur upon induction of a stimuli. A stimuli may comprise a change in chemical environment, light, temperature, pressure, electricity, or a combination thereof. A change in chemical environment may comprise a change in pH, a change in concentration, an addition of a denaturing agent, or a combination thereof. In some embodiments, a denaturing agent may comprise GuHCl, urea, sodium dodecyl sulfate (SDS), or a combination thereof. In some embodiments, upon decoupling, a biomolecule-analyte complex may enter and be a part of a supernant (e.g., the supernatant may comprise the biomolecule-analyte complex).

In some embodiments, an interaction between an analyte and a biomolecule may change the equilibrium of the interaction. For example, an interaction between an analyte and a biomolecule interacting with a surface may result in the biomolecule-analyte complex to decouple from the surface and enter the supernatant. On the other hand, for example, an interaction between an analyte and a biomolecule in the supernatant may result in the biomolecule-analyte complex to couple with the surface and exit the supernatant. In some embodiments, a dynamic equilibrium between a biomolecule-analyte complex coupled to or decoupled from the surface may exist or be reached over a period of time, given the conditions under which contacting may occur.

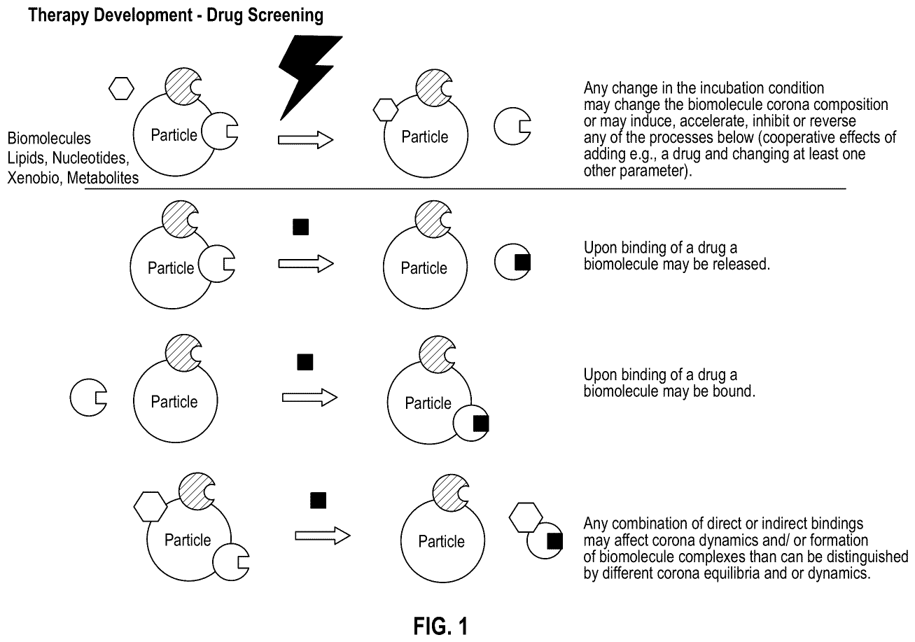

As an example, with reference to FIG. 1, a particle comprising a variety of biomolecules interacting with the particle may be exposed to one or more analytes. Upon forming a complex with an analyte, the biomolecule, for example, a protein, may undergo changes (e.g., folding, interactome, PTM decoration) and affect the binding equilibrium, thereby becoming destabilized and decoupling from the particle and may be released. The decoupled complex may become part of a supernatant. In some embodiments, as shown in FIG. 1, upon addition of an analyte (e.g., a drug), the analyte may complex with a biomolecule resulting in binding of the biomolecule-analyte complex. In other embodiments, as shown in FIG. 1, for example, any combination of direct or indirect bindings may affect corona dynamics and/or formation of biomolecule complexes that can be distinguished by different corona equilibria and or dynamics. In further embodiments, as shown in FIG. 1, changes in incubation condition may change the biomolecule corona composition or may induce, accelerate, inhibit, or reverse any of the forementioned processes.

### Systems

In one aspect, disclosed herein is an apparatus for identifying biomolecule-analyte interactions comprising: (i) a substrate comprising a plurality of partitions, where the plurality of partitions comprise a plurality of different biomolecules in contact with a surface to form one or more first biomolecule corona; (ii) a sample storage unit comprising the first biomolecule corona; and (iii) a loading unit that is movable at least across the substrate, where the loading unit transfers one or more volumes of an analyte in the sample storage unit to the plurality of partitions on the substrate, thereby contacting the one or more first biomolecule corona in the plurality of partitions with the analyte to form a second biomolecule corona and a supernatant.

The apparatus, in some embodiments, may be configured to perform any of the methods described herein. In some embodiments, the apparatus is automated. In some embodiments, an automated system may comprise a network of units with differentiated functions for isolating a biomolecule-analyte complex or an altered biomolecule corona, and the automated system is programed to perform a series of steps. In some aspects, the present disclosure provides an automated system comprising a network of units as described in WO2021/026172, which is incorporated herein by reference in its entirety. In some embodiments, the network of units may comprise differentiated functions in distinguishing biomolecule-analyte interactions using a plurality of biomolecule coronas: a first unit comprises a multichannel fluid transfer instrument for transferring fluids between units within the system; a second unit comprises a support for storing a plurality of biomolecules; a third unit comprises a support for a sensor array plate possessing partitions that comprise a plurality of biomolecule coronas for interacting with a population of analytes; a fourth unit comprises supports for storing a plurality of reagents; a fifth unit comprises supports for storing a reagent to be disposed of; a sixth unit comprises supports for storing consumables used by the multichannel fluid transfer instrument; and wherein the system is programed to perform a series of steps comprising: contacting a biological sample with a specified partition of the sensor array; incubating the biological sample with an analyte contained within the partition of the sensor array plate to form a biomolecule corona; removing a supernatant from a partition; and optionally preparing biomolecules from the biomolecule corona for analysis, such as mass spectrometry.

In some embodiments, the system is configured to incubate the same biological sample in different partitions of the sensor array plate having different concentrations of the analyte.

In some embodiments, the first unit comprises a degree of mobility that enables access to all other units within the system. In some embodiments, the first unit comprises a capacity to perform pipetting functions.

In some embodiments, the support of the second and/or third unit comprises support for a single plate, a 6 well plate, a 12 well plate, a 96 well plate, or a rack of microtubes. In some embodiments, the second and/or unit comprises a thermal unit capable of modulating the temperature of said support and a sample. In some embodiments, the second and/or third unit comprises a rotational unit capable of physically agitating and/or mixing a sample.

In some embodiments, the fourth unit comprises a set of reagents for: generating the sensor array plate; washing an unbound sample (e.g., analytes); and/or preparing a sample (e.g., of biomolecule coronas or a supernatant) for mass spectrometry. In some embodiments, contacting the biomolecule corona with a specified partition of the sensor array comprises pipetting a specified volume of the analyte into the specific partition of the sensor array. In some embodiments, contacting the biomolecule corona with a specified partition of the sensor array comprises pipetting a volume corresponding to a 1:1, 1:2:1:3, 1:4, 1:5, 1:6, 1:7, 1:8, 1:9, 1:10, 1:15, or 1:20 ratio of a analytes in a solution to the biomolecule coronas.

In some embodiments, contacting the biomolecule coronas with a specified partition of the sensor array comprises pipetting a volume of at least 10 microliters, at least 20 microliters at least 50 microliters, at least 100 microliters, at least 250 microliters, at least 500 microliters, or at least 1000 microliters of the biomolecule coronas into the specific partition of the sensor array. In some embodiments, contacting the biomolecule corona with a specified partition of the sensor array comprises pipetting a volume of no more than 1000 microliters, no more than 500 microliters, nor more than 250 microliters, no more than 150 microliters, no more than 100 microliters, no more than 75 microliters, no more than 50 microliters, or no more than 30 microliters.

In some embodiments, one or more volumes of an analyte may comprise one or more concentrations of the analyte. For example, a first volume may comprise a first concentration and a second volume may comprise a second concentration, where the first volume and the second volume are different and the first concentration and the second concentration are different. For example, a first volume may comprise a first concentration and a second volume may comprise a second concentration, where the first volume and the second volume are same and the first concentration and the second concentration are different. For example, a first volume may comprise a first concentration and a second volume may comprise a second concentration, where the first volume and the second volume are different and the first concentration and the second concentration are same.

In some embodiments, a substrate may comprise one or more partitions. In some embodiments, a substrate may comprise one or more wells. In some embodiments, a substrate may be a multi-well plate.

In some embodiments, a loading unit may comprise one or more pipettes. In some embodiments, a loading unit may comprise two or more pipettes (e.g., a plurality of pipettes). In some embodiments, a pipette in a loading unit may be configured to dispense a predetermined volume of solution (e.g., a solution comprising analyte) into a partition of the substrate. In some embodiments, a loading unit may comprise a plurality of pipettes and each pipette may be configured to dispense a predetermined, different volume of solution into a plurality of partitions of the substrate. In some embodiments, a loading unit may be configured to dispense a volume of about 1 microliter (μL) to about 1000 μL. In some embodiments, a loading unit may be configured to dispense a volume of about 5 microliter (μL) to about 1000 μL. In some embodiments, a loading unit may be configured to dispense a volume of about 100 microliter (μL) to about 1000 μL.

### Software

In one aspect, disclosed herein is a computer-based system for identifying a biomolecule-analyte interaction comprising a processer; and a non-transitory computer readable medium comprising a software configured to cause the processor to: (i) receive an assay data from the non-transitory computer readable medium, wherein the assay data comprises biomolecule information for a plurality of different biomolecule coronas and/or supernatants from a sample, wherein the plurality of different biomolecule coronas comprise a plurality of biomolecules and a surface, and wherein the different biomolecule coronas are contacted with a different concentration of an analyte; and (ii) performing a statistical analysis on the assay data to identify the at least one biomolecule-analyte interaction.

In some embodiments, the concentration of an analyte may be at least 1 parts per billion (ppb), 5 ppb, 10 ppb, 50 ppb, 100 ppb, 200 ppb, 300 ppb, 400 ppb, 500 ppb, 600 ppb, 700 ppb, 800 ppb, 900 ppb, 1 parts per million (ppm), 5 ppm, 10 ppm, 50 ppm, 100 ppm, 200 ppm, 300 ppm, 400 ppm, 500 ppm, 600 ppm, 700 ppm, 800 ppm, 900 ppm, 1000 ppm, 1500 ppm, 2000 ppm, 2500 ppm, 3000 ppm, 4000 ppm, 5000 ppm, 10000 ppm, 100000 ppm, or more. In some embodiments, the concentration of an analyte may be at least 1 ppb.

In some embodiments, the identifying is completed at multiple concentrations to measure a binding constant of the biomolecule-analyte interaction. In some embodiments, the biomolecule is a protein. In some embodiments, the analyte is a protein. In other embodiments, the analyte is a drug. In some embodiments, the drug is an antibody, a biotherapeutic, or a chemotherapeutic biomolecule. In some embodiments, the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof. In some embodiments, the surface is a particle or bead. In some embodiments, the surface is a particle.

In some aspects, the statistic analysis may comprise analysis using the Student's t-test may be used to identify statistically significant variations in the assay data that can be associated with the analyte. This may be, in some embodiments, if the relative abundance measured in the corona or supernatant changes by for example 1.2×, 1.5×, 2×, 4×, 10×, 100× significantly (p-value<than 5%).

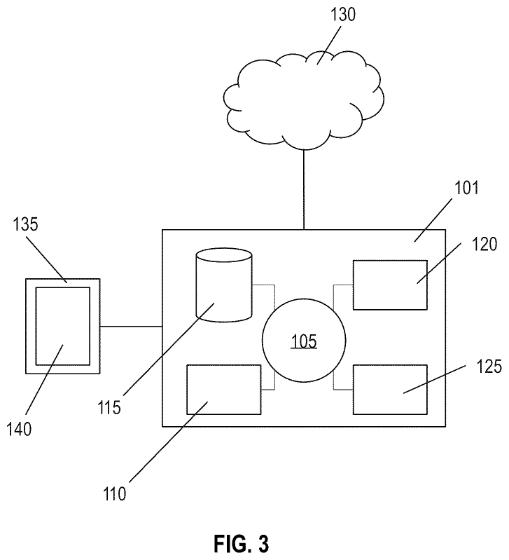

In an aspect, the present disclosure provides computer systems that are programmed or otherwise configured to implement methods of the disclosure, e.g., identifying biomolecule-analyte interactions. FIG. 3 shows a computer system 101 that is programmed or otherwise configured to implement a method for identifying biomolecule-analyte interactions. The computer system 101 may be configured to, for example, processing different assay data and detecting variations to identify biomolecule-analyte interactions as discussed elsewhere herein. The computer system 101 can be an electronic device of a user or a computer system that is remotely located with respect to the electronic device. The electronic device can be a mobile electronic device.

The computer system 101 may include a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are located external to the computer system 101 (e.g., on a remote server that is in communication with the computer system 101 through an intranet or the Internet).

The computer system 101 can communicate with one or more remote computer systems through the network 130. For instance, the computer system 101 can communicate with a remote computer system of a user (e.g., an operator overseeing or monitoring the fabrication of various film materials from waste cooking oil, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a portal for a user to monitor or track one or more processes for fabricating flexible film materials from waste cooking oil and compounds derived therefrom. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. For example, the algorithm may be configured to assign or sort interactions based on a class of biomolecule and/or analyte involved in the interaction.

### EMBODIMENTS

The following are exemplary embodiments of the disclosure herein:
1. A method for identifying a biomolecule-analyte interaction, the method comprising:
   contacting a surface with a first composition comprising first concentrations of a plurality of different biomolecules and a first concentration of one or more analytes to form one or more first biomolecule coronas;
   assaying the one or more first biomolecule coronas and/or a supernatant of the first composition to obtain a first assay data representing quantities of the plurality of different biomolecules in the one or more first biomolecule coronas and/or the supernatant of the first composition;
   providing a second assay data representing quantities of the plurality of different biomolecules in one or more second biomolecule coronas and/or a supernatant of a second composition; and
   identifying a biomolecule-analyte interaction based on one or more variations between the first assay data and the second assay data.
2. The method of embodiment 1, further comprising, (e) identifying structural changes in the biomolecule which are a result of the biomolecule-analyte interaction.
3. The method of embodiment 1, wherein in (c), the second composition comprises the first concentrations of the plurality of different biomolecules and optionally a second concentration of one or more analytes.
4. The method of embodiment 3, wherein the second composition comprises the first concentrations of the plurality of different biomolecules and no concentration of the one or more analytes.
5. The method of embodiment 3, wherein the second composition comprises the first concentrations of the plurality of different biomolecules and a second concentration of one or more analytes.
6. The method of embodiment 1, wherein in (c), the one or more second biomolecule coronas and the supernatant of the second composition are obtained by contacting the second composition with a second surface.
7. The method of embodiment 1, wherein in (c), the first surface and the second surface have the same physicochemical properties.
8. The method of embodiment 7, wherein the physicochemical properties are selected from the group consisting of composition, core material, shell material, porosity size, surface charge, hydrophobicity, hydrophilicity, surface functionality, surface topography, surface curvature, shape, or any combination thereof.
9. The method of embodiment 1, wherein in (c), the first concentration and the second concentration are different.
10. The method of any one of embodiments 1-9, wherein the second assay data is retrieved from a database.
11. The method of any one of embodiments 1-9, wherein the second assay data is acquired experimentally.
12. The method of any one of embodiments 1-11, wherein (c) is performed prior to (b).
13. The method of any one of embodiments 1-11, wherein (c) is performed prior to (a).
14. The method of any one of embodiments 1-11, wherein (c) and (a) are performed, at least in part, during the same time.
15. The method of any one of embodiments 1-14, wherein the plurality of different biomolecules comprise a nucleic acid, a small molecule, a protein, a lipid, a polysaccharide, a metabolite, or any combination thereof.
16. The method of embodiment 15, wherein a biomolecule of the plurality of different biomolecules is a nucleic acid, a small molecule, a protein, a lipid, a polysaccharide, or a metabolite.
17. The method of embodiment 16, wherein the biomolecule is a protein.
18. The method of embodiment 17, wherein the protein is a shuttle protein, a ligand, a member of a protein complex, comprises a low complexity region, comprises an interaction domain, involved in signal transduction, or any combination thereof.
19. The method of any one of embodiments 1-18, wherein the one or more first biomolecule coronas comprise one or more different biomolecules.
20. The method of any one of embodiments 1-19, wherein the one or more first biomolecule coronas comprise the one or more analytes.
21. The method of any one of embodiments 1-18, wherein the plurality of different biomolecules adsorb onto the surface to form the one or more first biomolecule coronas.
22. The method of any one of embodiments 1-18, wherein the one or more analytes adsorb onto the surface to form the one or more first biomolecule coronas.
23. The method of any one of embodiments 1-22, wherein the surface is a particle or bead.
24. The method of embodiment 23, wherein the surface is a particle.
25. The method of embodiment 24, wherein the particle is a micelle, liposome, iron oxide particle, silver particle, gold particle, palladium particle, quantum dots, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle,, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, and 2-(3-aminopropylamino)ethanol particle, protein functionalized particle, ubiquitin functionalized particle, polysaccharide coated particle, or dextran functionalized particle.
26. The method of embodiment 24, wherein the particle is a nanoparticle or a microparticle.
27. The method of embodiment 24, wherein the particle is a magnetic particle.
28. The method of embodiment 27, wherein the magnetic particle is a superparamagnetic iron oxide particle.
29. The method of embodiment 24, wherein the particle comprises iron oxide material.
30. The method of embodiment 29, wherein the particle comprises an iron oxide core.
31. The method of embodiment 29, wherein the particle has iron oxide crystals embedded in a polystyrene core.
32. The method of embodiment 24, wherein the particle comprises a polymer coating.
33. The method of embodiment 32, wherein the particle comprises a carboxylated polymer, an aminated polymer, a zwitterionic polymer, or any combination thereof.
34. The method of embodiment 24, wherein the particle comprises an iron oxide core with a silica shell coating.
35. The method of embodiment 24, wherein the particle comprises an iron oxide core with a poly(N-(3-(dimethylamino)propyl) methacrylamide) (PDMAPMA) coating.
36. The method of embodiment 24, wherein the particle comprises an iron oxide core with a poly(oligo(ethylene glycol) methyl ether methacrylate) (POEGMA) coating.
37. The method of embodiment 24, wherein the particle comprises a positive surface charge.
38. The method of embodiment 24, wherein the particle comprises a negative surface charge.
39. The method of embodiment 24, wherein the particle comprises a neutral surface charge.
40. The method of embodiment 1, wherein a biomolecule corona of the one or more first biomolecule coronas or the one or more second biomolecule coronas comprises:
   a first physicochemical property selected from the group consisting of a magnetic core, a polystyrene core, a metal core, a gold core, a metal oxide core, an iron oxide core, a polymeric core, and a silica core; and
   a second physicochemical property selected from the group consisting of a carboxylated surface, an amino surface, a silica surface, a polymer surface, a phosphate sugar functionalized surface, a phenol functionalized surface, a citrate functionalized surface, a Jeffamine surface, and a silica silanol surface.
41. The method of any one of embodiments 1-40, wherein the analyte is provided in a biological sample.
42. The method of embodiment 41, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.
43. The method of embodiment 42, wherein the biological sample comprises at least 100 proteins or protein groups.
44. The method of any one of embodiments 42-43, wherein the biological sample comprises at least 500 proteins or protein groups.
45. The method of any one of embodiments 42-44, wherein the biological sample comprises at least 1000 proteins or protein groups.
46. The method of any one of embodiments 1-45, wherein the analyte is not provided in a biological sample.
47. The method of any one of embodiments 1-45, wherein the analyte is a drug, therapeutic agent, nutrient, biomolecule, pathogen, or a metabolite thereof.
48. The method of embodiment 47, wherein the drug comprises an antibody, a biotherapeutic, or a chemotherapeutic biomolecule.
49. The method of embodiment 47 or 48, wherein the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof.
50. The method of embodiment 47, wherein the pathogen comprises a Hepatitis B virus, a Hepatitis C virus, a COVID-19 virus, or a HIV.
51. The method of any one of embodiments 1-45 the analyte is a non-naturally occurring small molecule.
52. The method of embodiment 51, wherein the small molecule comprises a drug.
53. The method of embodiment 47, wherein the analyte is a biomolecule.
54. The method of embodiment 53, wherein the biomolecule is a protein or a portion thereof.
55. The method of embodiment 54, wherein the protein comprises a receptor or enzyme configured to bind to the biomolecule coupled to the one or more distinct biomolecule coronas.
56. The method of embodiment 54, wherein the protein comprises a peptide sequence having a minimum length of 7 amino acid residues.
57. The method of embodiment 49, wherein the biomolecule is a lipid.
58. The method of embodiment 57, wherein the biomolecule is metabolite.
59. The method of any one of embodiments 1-58, wherein the analyte is configured to interact with one or more different biomolecules.
60. The method of embodiment 59, wherein the biomolecule of the one or more different biomolecules is a protein.
61. The method of embodiment 60, wherein the analyte interacts with an allosteric site on the protein.
62. The method of embodiment 61, wherein the analyte interacts with an active site on the protein.
63. The method of embodiment 62, wherein the analyte interacts with a target receptor site on the protein.
64. The method of any one of embodiments 59-63, wherein the interaction between a biomolecule of the one or more different biomolecules and the analyte comprises a coulombic interaction, a hydrogen bond, a Van der Waals interaction, solvent exclusion, hydrophobic effect, or a combination thereof.
65. The method of any one of embodiments 1-64, wherein the analyte comprises two or more binding moieties.
66. The method of embodiment 65, wherein a first binding moiety of the two or more binding moieties forms a complex with a first biomolecule of the plurality of different biomolecules and a second binding moiety of the two or more binding moieties forms a complex with a second biomolecule of the plurality of different biomolecules.
67. The method of embodiment 66, wherein the first biomolecule and the second biomolecule are different.
68. The method of embodiment 65, wherein a first binding moiety of the two or more binding moieties forms a first complex with a first component of the biomolecule of the one or more distinct biomolecule coronas and a second binding moiety of the two or more binding moieties forms a second complex with a second component of the biomolecule.
69. The method of embodiment 68, wherein the first component and the second component are different.
70. The method of embodiment 68 or 69, wherein the first complex and the second complex are in equilibrium.
71. The method of embodiment 65, wherein the analyte forms a complex with only one biomolecule of the plurality of different biomolecules.
72. The method of embodiment 71, wherein each of the two or more binding moieties simultaneously form a complex with the first biomolecule and the second biomolecule of the plurality of different biomolecules.
73. The method of any one of embodiments 1-72, wherein the biomolecule-analyte interaction is direct.
74. The method of any one of embodiments 1-72, the biomolecule-analyte interaction is indirect.
75. The method of any one of embodiments 1-72, wherein the biomolecule-analyte interaction comprises an electrostatic interaction between the biomolecule and the analyte.
76. The method of embodiment 75, wherein the electrostatic interaction comprises an ionic bond between the biomolecule and the analyte.
77. The method of embodiment 75, wherein the electrostatic interaction comprises a hydrogen-bond between the biomolecule and the analyte.
78. The method of embodiment 75, wherein the electrostatic interaction comprises Van der Waals forces.
79. The method of any one of embodiments 75-78, wherein the electrostatic interaction is reversible.
80. The method of any one of embodiments 1-79, wherein in (a) contacting comprises incubating the one or more first biomolecule coronas and the analyte.
81. The method of embodiment 80, wherein incubating is performed at a temperature of about 0 degrees Celsius to about 90 degrees Celsius.
82. The method of embodiment 80 or 81, wherein incubating is performed for a length of time of about 30 seconds to about 12 hours.
83. The method of any one of embodiments 1-82, wherein in (a) contacting comprises adjusting a pH of a mixture comprising the one or more first biomolecule corona.
84. The method of embodiment 83, wherein the pH is adjusted such that a component of a biomolecule of the plurality of different biomolecules is inhibited from forming a biomolecule-analyte interaction.
85. The method of any one of embodiments 1-84, wherein prior to (b) and subsequent to (a), the method comprises isolating the one or more first biomolecule coronas and/or the supernatant.
86. The method of embodiment 85, wherein isolating comprises magnetically isolating the one or more first biomolecule coronas.
87. The method of any one of embodiments 1-86, wherein in (b) assaying comprises a denaturing and/or chemical treatment.
88. The method of embodiment 87, wherein denaturing and/or chemical treatment comprises heating, reduction, alkylation, protease digestion, or combination thereof.
89. The method of any one of embodiments 1-88, wherein in (b) assaying comprises identifying protein groups.
90. The method of embodiment 89, wherein the assaying is capable of identifying 1 to 20,000 protein groups.
91. The method of embodiment 89, wherein at least 100 protein groups are identified.
92. The method of any one of embodiments 89-91, wherein assaying comprises centrifuging the supernatant comprising the biomolecule-analyte interaction.
93. The method of any one of embodiments 89-92, wherein assaying comprises detecting at least one of the one or more first biomolecule coronas or the biomolecule-analyte interactions in a sample through Mass Spectrometry.
94. The method of any one of embodiments 89-92, wherein the assaying comprises detecting at least one of the one or more first biomolecule coronas or the biomolecule-analyte interactions in a sample using high throughput single molecule protein sequencing.
95. The method of any one of embodiments 1-93, wherein in (b) assaying comprises quantifying at least one of the one or more first biomolecule coronas or the biomolecule-analyte interactions in a sample through Mass Spectrometry.
96. The method of any one of embodiments 1-93, wherein in (b) assaying comprises digesting proteins in the protein corona using a non-selective proteinase.
97. The method of embodiment 96, wherein the non-selective protease is Proteinase K.
98. The method of any one of embodiments 1-84, wherein in (b) assaying comprises cross-linking proteins in the protein corona and detecting cross-link proteins.
99. The method of embodiment 98, wherein the detecting comprises cross-linking mass spectrometry
100. The method of embodiment 98, wherein the cross-linking is performed with a mass spectrometry cleavable cross-linking agent.
101. The method of any one of embodiments 1-100, wherein the method further comprises repeating incubating, isolating, and assaying.
102. The method of embodiment 101, wherein the incubating, the isolating, and the assaying yields a percent quantile normalized coefficient (QNCV) of variation of 30% or less, as determined by comparing a mass spectrometry feature.
103. The method of embodiment 101, wherein the incubating, the isolating, and the assaying yields a percent quantile normalized coefficient (QNCV) of variation of 20% or less, as determined by comparing a mass spectrometry feature.
104. The method of any one of embodiments 1-99, wherein the method further comprises (e) detecting variations across one or more samples.
105. The method of embodiment 104, wherein detecting variations comprises detecting a change in a composition of the supernatant in comparison to an original composition of the supernatant.
106. The method of embodiment 105, wherein the original composition of the supernatant comprises the surface with the first composition comprising first concentrations of the plurality of different biomolecules without contact with the analyte.
107. The method of embodiment 105, wherein subsequent to (a) the supernatant comprises the biomolecule-analyte interaction.
108. The method of embodiment 104, wherein detecting variations comprises detecting a change in a composition of the one or more second biomolecule coronas in comparison to a composition of the one or more first biomolecule coronas.
109. The method of embodiment 108, wherein the composition of the one or more second biomolecule coronas comprises the plurality of different biomolecules without contact with the analyte.
110. The method of embodiment 108, wherein subsequent to (a) the one or more first biomolecule coronas is altered such that a biomolecule of the biomolecule-analyte interaction is absent from at least one of one or more first biomolecule coronas.
111. The method of embodiment 104, wherein detecting variations across one or more samples comprises detecting a change in a composition of the supernatant in comparison to an original composition of the supernatant and detecting a change in a composition of the one or more distinct biomolecule coronas in comparison to an original composition of the one or more distinct biomolecule coronas.
112. The method of embodiment 111, wherein the second composition of the supernatant comprises the one or more biomolecule coronas without contact with the analyte.
113. The method of embodiment 111, wherein subsequent to (a) the supernatant comprises the biomolecule-analyte interaction.
114. The method of embodiment 111, wherein the original composition of the one or more distinct biomolecule coronas comprises the one or more biomolecule coronas without contact with the analyte.
115. The method of embodiment 111, wherein subsequent to (a) the one or more distinct biomolecule coronas is altered such that a biomolecule of the biomolecule-analyte interaction is absent from at least one of one or more distinct biomolecule coronas.
116. The method of embodiment 1, wherein identifying the biomolecule-analyte interaction comprises identifying a biological state.
117. The method of embodiment 116, wherein the biological state is a healthy biological state.
118. The method of embodiment 116, wherein the biological state is a disease biological state.
119. The method of embodiment 1, wherein identifying the biomolecule-analyte interaction comprises identifying a signal transduction pathway.
120. The method of any one of embodiments 1-118, wherein a thermodynamic stability of the biomolecule-analyte interaction is greater than a thermodynamic stability of an interaction comprising the analyte and the surface with a first composition such that the biomolecule-analyte interaction decouples from the one or more first biomolecule coronas, and wherein a composition of the one or more first biomolecule coronas is altered.
121. The method of embodiment 120, wherein the biomolecule-analyte interaction decouples from the one or more first biomolecule coronas spontaneously.
122. The method of embodiment 120, wherein the biomolecule-analyte interaction decouples from the one or more first biomolecule coronas upon induction of a stimuli.
123. The method of embodiment 122, wherein the stimuli is a change in chemical environment, light, temperature, pressure, electricity, or a combination thereof.
124. The method of embodiment 123, wherein the change in chemical environment comprises a change in pH, a change in concentration, an additional of a denaturing agent, or a combination thereof.
125. The method of embodiment 124, wherein the denaturing agent comprises GuHCl, urea, SDS, or a combination thereof.
126. The method of any one of embodiments 120-124 upon decoupling, the biomolecule-analyte interaction is a part of the supernatant.
127. The method of any one of embodiments 1-125, wherein the method comprises contacting two or more biomolecule coronas with the analyte, wherein each distinct biomolecule corona of the two or more distinct biomolecule coronas differs from each other by one or more physicochemical properties.
128. The method of embodiment 127, wherein the one or more physicochemical properties are selected from the group consisting of: composition, size, surface charge, hydrophobicity, hydrophilicity, surface functionality, surface topography, surface curvature, shape, and any combination thereof.
129. The method of any one of embodiments 1-128, wherein in (b), the method comprises contacting two or more analytes with the biomolecule coronas, wherein each analyte of the two or more analyte is different.
130. The method of embodiment 129, wherein each analyte of the two or more analyte forms a complex with a biomolecule coupled to the surface.
131. An apparatus for identifying biomolecule-analyte interactions, the apparatus comprising:
   a substrate comprising a plurality of partitions, wherein the plurality of partitions comprises plurality of different biomolecules contacted with a surface to form one or more first biomolecule corona;
   a sample storage unit comprising the first biomolecule corona; and
   a loading unit that is movable at least across the substrate;
wherein the apparatus is programmed to perform a series of steps comprising:
   contacting a biological sample with a specified partition of the sensor array;
   incubating the biological sample with an analyte contained within the partition of the sensor array plate to form a biomolecule corona; removing a supernatant from a partition; and optionally preparing biomolecules from the biomolecule corona for analysis.
132. The apparatus of embodiment 131, wherein the apparatus is automated.
133. The apparatus of any one of embodiments 131-132, wherein the one or more volumes of an analyte comprise one or more concentrations of the analyte.
134. The apparatus of any one of embodiments 131-133, wherein the substrate is a multi-well plate.
135. The apparatus of any one of embodiments 131-134, wherein the loading unit comprises a plurality of pipettes.
136. The apparatus of embodiment 135, wherein the loading unit is configured to dispense a volume of 5 μL to 1000 μL of a solution into one or more partitions of the plurality of partitions.
137. The apparatus of embodiment 136, wherein the volume of a solution dispensed into each of the one or more partitions of the plurality of partitions is different.
138. The apparatus of any one of embodiments 131-137, wherein the analyte comprises protein.
139. The apparatus of any one of embodiments 131-137, wherein the analyte comprises a drug.
140. The apparatus of any one of embodiments 131-139, wherein the second biomolecule corona is assayed.
141. The apparatus of any one of embodiments 131-140, wherein the supernatant is assayed.
142. A computer-based system for identifying a biomolecule-analyte interaction, the system comprising:
   A Processor, and
   a non-transitory computer readable medium comprising a software configured to cause the processor to:
   (i) receive an assay data from the non-transitory computer readable medium, wherein the assay data comprises biomolecule information for a plurality of different biomolecule coronas and/or supernatants from a sample, wherein the plurality of different biomolecule coronas comprise a plurality of biomolecules and a surface, and wherein the different biomolecule coronas are contacted with a different concentration of an analyte; and
   (ii) performing a statistical analysis on the assay data to identify the at least one biomolecule-analyte interaction.
143. The system of embodiment 142, wherein the concentration of an analyte is at least 1 ppb.
144. The system of embodiment 142 or 143, wherein the identifying is completed at multiple concentrations to measure a binding constant for the biomolecule-analyte interaction.
145. The system of any one of embodiments 142-144, wherein the biomolecule is a protein.
146. The system of any one of embodiments 142-145, wherein the analyte is a protein.
147. The system of any one of embodiments 142-145, wherein the analyte is a drug.
148. The system of embodiment 147, wherein the drug is an antibody, a biotherapeutic, or a chemotherapeutic biomolecule.
149. The method of embodiment 148, wherein the drug comprises a methamphetamine, isotretinoin, an antibiotic, an anti-platelet medication, dutasteride, a blood thinner, insulin, hepatitis B immune globulin, a growth hormone, tamsulosin, finasteride, acitretin, etretinate, or any combination thereof.
150. The system of any one of embodiments 142-149, wherein the surface is a particle or bead.
151. The system of embodiment 150, wherein the surface is a particle.
152. A method of identifying biomolecule-analyte interactions in a subject, the method comprising:
   contacting a first surface with a first composition comprising concentrations of a plurality of different biomolecules and a first concentration of one or more analytes to form one or more first biomolecule corona, wherein the plurality of different biomolecules are derived from a biological sample of the subject;
   assaying the one or more first biomolecule coronas and/or a supernatant of the first composition to obtain a first assay data representing quantities of the biomolecules in the first biomolecule coronas and/or the supernatant of the first composition;
   contacting a second surface with a second composition comprising concentrations of a plurality of different biomolecules and a second concentration of one or more analytes to form one or more second biomolecule corona, wherein the plurality of different biomolecules are derived from the biological sample of the subject
   assaying the one or more second biomolecule coronas and/or a supernatant of the second composition to obtain a second assay data representing quantities of the biomolecules in the second biomolecule coronas and/or the supernatant of the second composition; and
   identifying biomolecules that interact with the analytes based on variations between the first assay data and second assay data.
153. The method of embodiment 152, wherein the first composition and the second composition are different.
154. The method of embodiment 153, wherein the first concentration of the one or more analytes is different from the second concentration of the one or more analytes.
155. The method of any one of embodiments 152-154, wherein the first surface and the second surface are the same.
156. The method of any one of embodiments 152-155, wherein first surface and the second surface is a particle or bead.
157. The method of embodiment 156, wherein the surface is a particle.
158. The method of embodiment 157, wherein the particle is a nanoparticle or a microparticle.
159. The method of embodiment 157, wherein the particle is a magnetic particle.
160. The method of embodiment 159, wherein the magnetic particle is a superparamagnetic iron oxide particle.
161. The method of embodiment 157, wherein the particle comprises iron oxide material.
162. The method of embodiment 158, wherein the particle has iron oxide crystals embedded in a polystyrene core.
163. The method of embodiment 157, wherein the particle comprises a polymer coating.
164. The method of embodiment 160, wherein the particle comprises a carboxylated polymer, an aminated polymer, a zwitterionic polymer, or any combination thereof.
165. The method of embodiment 157, wherein the particle comprises an iron oxide core with a silica shell coating.
166. The method of embodiment 157, wherein the particle comprises an iron oxide core with a poly(N-(3-dimethylamino)propyl) methacrylamide) (PDMAPMA) coating.
167. The method of embodiment 157, wherein the particle comprises an iron oxide core with a poly(oligo(ethylene glycol) methyl ether methacrylate) POEGMA) coating.
168. The method of any one of embodiments 157-167, wherein the particle comprises a positive surface charge.
169. The method of any one of embodiments 157-167, wherein the particle comprises a negative surface charge.
170. The method of any one of embodiments 157-167, wherein the particle comprises a neutral surface charge.
171. The method of any one of embodiments 152-170, wherein the analyte is a drug or a therapeutic agent.
172. The method of any one of embodiments 152-171, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.
173. The method of embodiment 172, wherein the biological sample is a biofluid.
174. The method of embodiment 172, wherein the biological sample is plasma or serum.
175. A method comprising:
   incubating a biofluid with a surface and an analyte to form a protein corona on the surface;
   analyzing the proteins from the protein corona using mass spectrometry to obtain a protein corona signature associated with the biofluid and analyte; and
   comparing the protein corona to a suitable control protein corona signature to identify at least one protein-analyte interaction.
176. The method of embodiment 175, wherein the biofluid is plasma or serum.
177. The method of any one of embodiments 175-176, wherein the surface comprises a plurality of particles.
178. The method of any one of embodiments 175-177, wherein incubating the biofluid with the surface and the analyte comprises incubating for at least 20 minutes.
179. The method of any one of embodiments 175-178, wherein incubating the biofluid with the surface and the analyte comprises incubating at a temperature of 15° C. to 75° C.
180. The method of any one of embodiments 175-179, wherein the proteins from the protein corona are digested before analysis.
181. The method of any of embodiments 175-180, further comprising separating the protein corona and surface from a supernatant before analyzing the protein corona.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Analyzing Protein-Drug Interactions in Plasma

Plasma samples are analyzed using a modification of the PROTEOGRAPH v1.2 workflow to analyze protein-drug interactions. Briefly, 40 microliters of plasma sample is aliquoted into separate wells containing nanoparticles. Different concentrations of one or more analytes, such as warfarin, heparin, or naproxen, are added to each of the wells and incubated to form a protein corona on the nanoparticles and then the protein corona processed to perform LC-MS analysis. The LC-MS data can be analyzed to identify changes in the protein corona signature that can be associated with drug interactions.

### Example 2: Analyzing Protein-DRUG Interactions in Plasma during Washing Steps

The experiment in Example 1 is repeated, except that the drug is not included during the initial incubation. The drug is instead included in a solution that is incubated with the protein corona on the nanoparticle after removing the supernatant of the plasma sample. The LC-MS results can be analyzed to identify changes in the protein corona signature that can be associated with drug interactions that occurred during the washing step.

### Example 3: Analyzing Protein-Drug Interactions in Plasma Using Non-Selective Protease

The experiment in Example 1 is repeated, except during processing of the protein corona to perform LC-MS, the protein corona is exposed to Proteinase K while the protein corona is attached to the nanoparticle. The protein corona may then be digested using a selective protease, such as trypsin. The LC-MS results can be analyzed to identify changes in the protein corona signature that can be associated with drug interactions that occurred during the washing step. In particular, the peptides detected will vary based on protein accessibility to Proteinase K digestion while in the protein corona, which can provide additional insight into potential binding interactions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby. The section headings used herein are for organizational purposes only and are not to be construed as limiting the subject matter described.

1.-45. (canceled)

46. A method for identifying a biomolecule-analyte interaction, the method comprising:
contacting a first surface with a first composition comprising a plurality of different biomolecules and a first concentration of an analyte to form one or more first biomolecule coronas;
assaying the one or more first biomolecule coronas and/or a supernatant of the first composition to obtain a first assay data representing abundances of the plurality of different biomolecules in the one or more first biomolecule coronas and/or the supernatant of the first composition;
providing a second assay data representing abundances of the plurality of different biomolecules in one or more second biomolecule coronas and/or a supernatant of a second composition; and
identifying a biomolecule-analyte interaction based on one or more variations between the first assay data and the second assay data.

47. The method of claim 46, wherein the second composition comprises the plurality of different biomolecules and optionally a second concentration of the analyte.

48. The method of claim 47, wherein the second composition comprises the plurality of different biomolecules and no concentration of the analyte.

49. The method of claim 47, wherein the second composition comprises the plurality of different biomolecules and a second concentration of analyte.

50. The method of claim 46, wherein the one or more second biomolecule coronas and the supernatant of the second composition are obtained by contacting the second composition with a second surface.

51. The method of claim 46, wherein the second assay data is retrieved from a database.

52. The method of claim 46, wherein the plurality of different biomolecules comprises proteins.

53. The method of claim 46, wherein the first surface is a particle or bead.

54. The method of claim 53, wherein the particle is a magnetic particle, wherein the magnetic particle is a superparamagnetic iron oxide particle.

55. The method of claim 53, wherein the particle comprises a polymer or silica shell coating.

56. The method of claim 46, wherein the first composition or the second composition is a biological sample, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

57. The method of claim 56, wherein the biological sample comprises at least 100 proteins or protein groups.

58. The method of claim 46, wherein the analyte is a drug, therapeutic agent, nutrient, biomolecule, pathogen, or a metabolite thereof.

59. The method of claim 46, wherein the contacting comprises adjusting a pH of a mixture comprising the one or more first biomolecule corona.

60. The method of claim 59, wherein the pH is adjusted such that a component of a biomolecule of the plurality of different biomolecules is inhibited from forming a biomolecule-analyte interaction.

61. The method of claim 46, wherein the assaying comprises identifying protein groups by mass spectrometry.

62. The method of claim 46, wherein the assaying comprises digesting proteins in the biomolecule corona using a non-selective proteinase.

63. The method of claim 46, wherein assaying comprises cross-linking proteins in the biomolecule corona and detecting cross-linked proteins.

64. The method of claim 46, wherein identifying the biomolecule-analyte interaction comprises identifying a biological state.

65. The method of claim 46, wherein the method comprises contacting two or more biomolecule coronas with the analyte, wherein each distinct biomolecule corona of the two or more distinct biomolecule coronas differs from each other by one or more physicochemical properties.
